(19) United States
(12) Patent Application Publication
(10) Pub. No.: US 2026/0165978 A1
(43) Pub. Date: Jun. 18, 2026

(54) METHODS FOR ACTIVELY LOADING THERAPEUTIC MOLECULES INTO MODIFIED EXTRACELLULAR VESICLES FOR TARGETED DELIVERY TO CELLS

(71) Applicant: Mercury Bio, Inc., Santa Fe, NM (US)

(72) Inventors: Richard T. Sayre, Los Alamos, NM (US); Tatiana Vinogradova-Shah, Rochester, NY (US); Elena Sineva, Bernalillo, NM (US); Callum Bell, Santa Fe, NM (US); Arthur Leo Kruckeberg, Santa Fe, NM (US)

(21) Appl. No.: 19/119,000

(22) PCT Filed: Oct. 7, 2023
(86) PCT No.: PCT/US2023/076329

§ 371 Date: Apr. 7, 2025

Related U.S. Application Data
(60) Provisional application No. 63/414,317, filed on Oct. 7, 2022.

(51) Int. Cl. A61K 9/50; A61K 31/09; A61K 31/165; A61K 31/352; A61K 31/404; A61K 31/473; A61K 31/496; A61K 31/505; A61K 31/513; A61K 31/5383; A61K 31/7036; A61K 31/704; A61K 31/7048; A61K 45/06

(52) U.S. Cl. CPC A61K 9/5089; A61K 9/5068; A61K 31/09; A61K 31/165; A61K 31/352; A61K 31/404; A61K 31/473; A61K 31/496; A61K 31/505; A61K 31/513; A61K 31/5383; A61K 31/7036; A61K 31/704; A61K 31/7048; A61K 45/06

(57) ABSTRACT

Methods and compositions for actively loading of therapeutic compositions into yeast-generated extracellular vesicles (EVs) and their methods of use in treating one or more diseases or conditions.

### CROSS-REFERENCE TO RELATED APPLICATIONS

This International PCT application claims the benefit of and priority to U.S. Provisional Application No. 63/414,317 filed Oct. 7, 2022, the specification, claims and drawings of which are incorporated herein by reference in their entirety.

### SEQUENCE LISTING

The instant application contains contents of the electronic sequence listing (90355-00081-Sequence-Listing.xml; Size: 26,317 bytes; and Date of Creation: Oct. 5, 2023) is herein incorporated by reference in its entirety.

### TECHNICAL FIELD

The present invention is directed to novel methods, systems, and compositions to facilitate the active loading of therapeutic compositions into yeast-generated extracellular vesicles (yEVs or EVs) and their methods of use in treating one or more disease or conditions.

### BACKGROUND

One of the major challenges facing the use of therapeutic molecules and compounds, also sometimes referred to as a drugs, therapeutics, or therapeutic compositions, to control diseases and genetic disorders is an effective means to deliver the drug to a targeted tissue while avoiding degradation or its elimination from the body. Recently, lipid encased nanovesicles have proven to be an effective means to deliver therapeutic molecules to human cells while protecting their cargo from degradation. The use of lipid nanovesicles to deliver mRNA to cells has proven to be an efficient means to vaccinate humans against SARS-COV-2. However, the artificial lipids frequently used to make these nanovesicles are not tissue or cell specific and are not well tolerated by humans limiting their applications to non-therapeutic purposes.

An alternative, natural lipid nanovesicle that has also been shown to package RNA, proteins and other small molecules are extracellular vesicles (EVs) or exosomes produced and released from the surface of human cells. Most eukaryotic EVs are well tolerated but have limitations due to the heterogeneity of cell types from which they originate resulting in broad distributions of particle size and heterogeneous cargo. Furthermore, EVs or exosomes produced in human cell lines can potentially be contaminated by non-target RNA species and human pathogens such as viruses. In addition, each of these delivery platforms must be stored at ultralow temperatures and are costly to produce

An alternative platform for producing more uniform EVs that package therapeutic molecules is the production of EVs in single celled eukaryotic organisms (non-human) having uniform genetic traits. Examples of single-celled organisms that can be engineered to deliver therapeutic molecules include yeast, such as Saccharomyces cerevisiae and Saccharomyces boulardii. Significantly, these yeast strains are well tolerated by humans, being used in the manufacture of food and beverages consumed by humans. Significantly, S. boulardii has been safely used as a human probiotic for over 65 years. More specifically, S. boulardii has many traits that are ideal to produce EVs for delivery of therapeutics to humans. S. boulardii produces EVs that package RNA, proteins, and other small molecules. In addition, the genome of S. boulardii is available and S. boulardii can be genetically engineered and/or have its genome edited for the purpose of modifying EV targeting or cargo. Analyses of the transcriptome of S. boulardii whole cells indicates that there are no RNA species that have greater than 11 nucleotides similarity to human transcripts and there is only a single RNA species that has any complementary to a human transcript, a non-coding RNA species. This observation contrasts with human exosomes that contain 100s of RNA species that could have off-target effects. S. boulardii can also be grown at large scale in fermenters and has a shelf life of over a year at room temperature when freeze-dried. Finally, it has been observed that yeast extracellular vesicles can be delivered to mice orally as well as by injection and have access to all major organs in the mouse body. These traits open the possibility of using S. boulardii as a complete, single celled system for the large-scale production and packaging of therapeutic molecules into EVs for delivery to humans.

Several traditional methods have been developed to load therapeutic compositions into EVs for delivery to target cells or tissues. These methods for encapsulating cargo into EVs can be roughly divided into two types: cell-based loading methods and non-cell-based loading methods. In the cell-based loading approach, cargos are usually delivered into the donor cells first. After being packaged into EVs, the cargos can be secreted and collected in an EV-carrying manner for therapeutic use. Non-cell-based loading approaches involve directly loading chemicals or biomolecules into isolated EVs through electroporation, sonication, incubation, and/or transfection. However, each of these non-cell-based methods involve specific technical and commercial limitations. For example, passive incubation loading typically includes low loading efficiencies, while transfection relies on transfection efficiency, which is always variable, and may further alter the structure of the EVs. Methods such as electroporation, sonication, and freeze thaw disrupt the EVs structure and increase EV instability and can further result in undesired EV aggregation and low yields. Finally, current cell, and non-cell-based methods of EV loading lack the ability to precisely, selectively, and uniformly load each EV so that it can be more effectively dosed for therapeutic applications.

As such, there exists a long-felt need for an efficient, and commercially viable, method to load therapeutic compositions precisely and uniformly into EV structures, all while causing minimal disruption to their structure and aggregation patterns.

### SUMMARY OF THE INVENTION

The present invention is directed to novel methods, systems, and compositions for actively loading a xenobiotic, such as a therapeutic compound into an EV, wherein the EV can deliver the therapeutic composition to a target cell thereby treating a disease or condition or generating some physiological or other effect.

In another aspect, the present invention includes systems and compositions to genetically modify yeast cells, and preferably a Saccharomyces yeast cell, to express one or more heterologous peptides configured to facilitate the active transport of target compositions, such as therapeutic compositions into an EV derived from said yeast cell. In one preferred aspect the current invention includes novel constructs for the heterologous expression of one or more fusion peptides in yeast adapted to facilitate for active transport of therapeutic compositions into a yeast EV. In another aspect, the present invention includes systems and compositions for actively loading a xenobiotic, and preferably a hydrophobic xenobiotic compound into a Saccharomyces-generated extracellular vesicle (EV). In a preferred aspect, this embodiment can include generating an EV having a heterologous membrane bound fusion peptide. In this preferred aspect, the fusion peptide includes a first domain comprising membrane targeting motif, which can include an EV membrane protein anchor and/or a protein extracellular vesicle sorting motif (p-ESM), and a second domain comprising a proton driven xenobiotic transporter. The membrane targeting motif can be configured to orient the proton driven xenobiotic transporter such that when a pH gradient is established across the membrane of the EV, the transporter will actively and selectable transport a xenobiotic from the external environment into the lumen, or internal portion of the EV by the transporter. In a quantity of EV can be loaded with one or more xenobiotics, and preferably therapeutic compounds and isolated. These isolated EVs can be combined with a pharmaceutically acceptable carrier to form a pharmaceutical composition that can be administered to a subject in need thereof to treat a disease or condition.

In another aspect, the present invention includes a genetically modified yeast cell configured to express a heterologous nucleotide, operably linked to a promoter, encoding a fusion peptide. In this preferred aspect, the fusion peptide includes a first domain comprising membrane targeting motif, which can include an EV membrane protein anchor and/or a protein extracellular vesicle sorting motif (p-ESM), and a second domain comprising a proton driven xenobiotic transporter. The fusion peptide of the invention can be expressed in the yeast cell and incorporated into a yeast-derived EV through the membrane protein anchor. The EVs having the fusion peptide can be isolated and further configured to have an acidic lumenal pH. In this preferred aspect, the yeast-derived EVs of the invention can be contacted with a xenobiotic and a solution, preferably having a different pH than the pH of the lumen forming a pH gradient across the membrane of the EV, such that the xenobiotic is transported into the lumen of the EV by the transporter driven by the artificial gradient.

Another aspect of the invention includes methods and compositions for the transport and deconjugation of conjugated compositions, such as conjugated xenobiotics, through the co-expression of one or more deconjugating enzymes. Co-expression of a xenobiotic deconjugating enzyme in yeast may preferably be adapted to allow the deconjugating enzyme to be anchored to the lumenal side of the EV membrane. This aspect allows the system to regenerate the parent xenobiotic and to reduce loss of xenobiotic conjugates (glycosides, glutathione conjugates, etc.) from the EVs and to make the xenobiotic biologically more active than its conjugate.

Another aspect of the invention includes methods and compositions for the co-expression of receptor-specific ligands anchored to EV membrane proteins. In this preferred aspect, genetically modified yeast cells are adapted to heterologously express and present on the surface of EVs receptor-specific ligands anchored to EV membrane proteins as a fusion peptide.

Additional aspects of the invention will be evident from the specification, figures, and claims provided herein.

### BRIEF DESCRIPTION OF THE FIGURES

FIG. 1. Upper, yEV membrane protein model of the truncated Sur7-AbeM fusion protein. Truncated Sur7 is indicated in red and the AbeM protein in blue. Lower, AbeM-pESM protein with C-terminal yEV protein sorting motif used to localize the AbeM protein into the yEV membrane.

FIG. 2. Fluorescence excitation and emission spectra of doxorubicin in solution

FIG. 3. Calibration curves for doxorubicin absorbance (A) and fluorescence (B) are generated from data shown in FIG. 2. Despite a very low doxorubicin quantum yield, fluorescence is detected at a very low concentration of the drug (˜2 μM). The doxorubicin fluorescence signal was detected at λ550 nm using an excitation λ of 470 nm. Since the scattering of concentrated exosome suspensions is very high, proper detection of the doxorubicin signal requires spectrum taking and background subtraction.

FIG. 4. The efficiency of cellular uptake of free doxorubicin (DX) vs doxorubicin loaded into yEVs (EVs-DX) by electroporation (A) or by AbeM-mediated uptake (B).

FIG. 5. Properties of yEVs before and after acidification treatment.

FIG. 6. Oregon Green fluorescence decay from yEVs equilibrated at pH 3 and suspended in pH 8 buffer.

FIG. 7. Procedure for generating yEV transmembrane proton gradient and pH-dependent drug loading.

FIG. 8. Relative uptake of doxorubicin into wild type (WT) and engineered (Sur7-Tr and Sur7-Full) as a function of yEV lumenal pH.

FIG. 9. Determination of the optimal yEV luminal pH for doxorubicin uptake.

FIG. 10. Final comparison of doxorubicin uptake into various constructs.

FIG. 11. Protein structure of Saccharomyces cerevisiae T-SNARE EV membrane localized protein SSO2 predicted by Alphafold.

FIG. 12. Protein structure of Haloterrigena turkmenica rhodopsin predicted by Alphafold.

FIG. 13. Human glycophorin A protein structure predicted by Alphafold.

FIG. 14. AbeM protein structure predicted by Alphafold.

### DETAILED DESCRIPTION OF THE INVENTION

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference. The terminology used herein is for describing particular embodiments and is not intended to be limiting. As used herein, the singular forms “a,” “and” and “the” include plural referents unless the content and context clearly dictate otherwise. Thus, for example, a reference to “a” or “the” marker may include a combination of two or more such markers. Unless defined otherwise, all scientific and technical terms are to be understood as having the same meaning as commonly used in the art to which they pertain. For the purposes of the present invention, the following terms are defined above.

The present invention is directed to EVs derived from Saccharomyces, such as Saccharomyces cerevisiae or Saccharomyces boulardii engineered to actively load therapeutic compositions into an EV that can be administered to a subject in need thereof to treat a disease or condition.

In one aspect, the invention describes novel systems, methods and compositions actively loading therapeutic compositions, such as biological and non-biological therapeutic molecules, or conjugates of biological and/or non-biological compositions into yeast EVs using a light driven proton pump to drive transport by a proton-xenobiotic antiporter into a yeast EVs.

In one aspect, the invention describes novel systems, methods and compositions actively loading therapeutic compositions, such as biological and non-biological therapeutic molecules, or conjugates of biological and/or non-biological compositions into yeast EVs using a pH gradient to drive transport of therapeutic compositions, such as one or more xenobiotics, by an AbeM proton antiporter, into a yeast EVs.

In one aspect, the invention describes systems and methods for loading a xenobiotic compound into a Saccharomyces-generated extracellular vesicle (EV) engineered to include a heterologous membrane bound xenobiotic transporter. In a preferred embodiment, a yeast cell, and preferably a S. boulardii yeast cell can be transformed by an expression vector, operably linker to a promoter, encoding a heterologous fusion peptide having a first domain comprising a first membrane targeting domain and a second domain comprising a proton driven xenobiotic transporter.

In one preferred embodiment, the membrane targeting domain includes an EV membrane protein anchor. In this embodiment, the fusion peptide of the invention is configured such that the EV membrane protein is adapted to bind to the EV membrane and orient the xenobiotic transporter in relation to the EV membrane such that the target xenobiotic, such as a therapeutic compound is actively and specifically loaded from the external EV environment into the EV. In a preferred embodiment, the fusion peptide of the invention includes the amino acid sequence according to SEQ ID NO. 19, or a sequence having at least 80% sequence homology with SEQ ID NO. 19, or a fragment thereof.

In another preferred embodiment, the membrane targeting domain includes a protein extracellular vesicle sorting motif (p-ESM). As used herein, a p-ESM includes a protein sequence in a polypeptide that forms a structural motif that causes the corresponding polypeptide to be preferentially packaged when introduced to a yeast-derived EV. Exemplary p-ESMs are described in U.S. Patent Application No. 63/536,478, and specific p-ESMs of the invention are provided in Table 1, Table 1A; see also SEQ ID NO's. 5-458, and in a preferred embodiment SEQ ID NO's. 1-24, all of which are incorporated herein by specific reference. In a preferred embodiment, the fusion peptide of the invention includes the amino acid sequence according to SEQ ID NO. 21, or a sequence having at least 80% sequence homology with SEQ ID NO. 21, or a fragment thereof.

In one preferred embodiment, the fusion peptide of the invention includes a proton driven xenobiotic transporter such as an AbeM protein (SEQ ID NO. 4), or fragment thereof. As further described in the examples below, in this embodiment, the AbeM transporter can be coupled, preferably through a linker, and preferably a peptide linker according to SEQ ID NO. 20, with a second domain comprising a Sur7 protein that has been engineered to orient the AbeM transporter in relation to the outer membrane of the EV to pump the xenobiotics into the EV in response to a pH gradient between the internal and external EV environments. In a preferred embodiment, this modified Sur7 protein can include a truncated Sur7 protein according to SEQ ID NO. 17, or a sequence having at least 80% sequence homology with SEQ ID NO. 17, or a fragment thereof.

In another preferred embodiment, the fusion peptide of the invention includes the proton driven xenobiotic transporter, again such as an AbeM protein (SEQ ID NO. 4), or fragment thereof that is with a second domain comprising a p-ESM, that in a preferred embodiment preferentially directs the fusion peptide to a yEV, and can further orient the xenobiotic transporter in relation to the outer membrane of the EV such that it pumps a xenobiotic into the EV in response to a pH gradient between the internal and external EV environments. In a preferred embodiment, this modified p-ESM protein can include a peptide according to SEQ ID NO. 22, or a sequence having at least 80% sequence homology with SEQ ID NO. 2, or a fragment thereof. As noted above, additional exemplary p-ESMs are described in U.S. Patent Application No. 63/536,478, all of which are incorporated herein by specific reference.

As noted above, in one preferred embodiment, the EV of the invention can be configured to include acidic lumenal pH. In a preferred embodiment, this can be accomplished through one or more proton pumps, while in other preferred embodiments, this can further be accomplished by incubating the EVs in an acidic environment, such as an acidic buffer solution. In this embodiment, the range of the acidic solution, as well as the desired lumenal pH can be determined based on the target xenobiotic, quantity of EVs, as well as the desired dose to be actively loaded into the EV. In a preferred embodiment, the acidic solution can have a pH of approximately 3, which can generate an EV lumenal pH of approximately 4 after incubation. Naturally, this is an exemplary embodiment as the pH solution and lumenal pH can encompass a variety of pH ranges.

Next, the EV having an acidic lumenal pH can be contacted with an environment having a pH that is higher, or more basic that the lumenal pH generating a pH gradient across the membrane of the EV, which energetically drives the transporter to import a target xenobiotic from the external environment into the EV. In a preferred embodiment, the step of generating a pH gradient across the membrane of the EV includes contacting the EV with a transfer solution, sometimes referred to as a transfer media, having a pH that is higher than the lumenal pH. Again, while a range of pH gradients are contemplated within the scope of the invention, in one embodiment the transfer solution has a pH of approximately 8. As further described in the examples below, the xenobiotic can be contacted with the EV, for example by being added to the transfer media prior to, or after generating the pH gradient. The amount of xenobiotic to be loaded can be dependent on the transported used, the physical characteristic of the xenobiotic to be transported as well as the pH gradient generated to drive the active transport process.

The loaded EVs of the invention can further be isolated from the transfer media and combined with a pharmaceutically acceptable carrier forming a pharmaceutical composition as generally defined herein. A therapeutically effective amount of the pharmaceutical composition of the invention can be administered to a xenobiotic to a subject in need thereof, the subject preferably being a mammal, and more preferably being a human or human cell.

In another preferred aspect, the invention includes a genetically modified yeast cell configured to express a heterologous fusion peptide for actively loading a therapeutic composition into an EV. In this preferred embodiment, an exemplary EV membrane protein anchor, a light-dependent proton pump, optionally a transmembrane domain, which may be included depending on the orientation of the transporter, for example a proton driven drug transporter in the EV membrane; a proton-driven drug transporter and a linker protein. In another preferred embodiment, heterologous fusion peptide for actively loading a therapeutic composition into an EV may include an EV-localized Saccharomyces cerevisiae SSO2 membrane protein, which may be a full-length or a truncated portion extending from position M1 to D161 of SEQ ID NO. 1; 2) rhodopsin (BR) derived from Haloterrigena turkmenica 3) a human glycophorin A transmembrane protein to allow for the proper functional orientation of C-terminal protein fusions domains including BR and AbeM, 4) an AbeM protein from Acinetobacter baumannii and; 5) a linker peptide adapted to allow proper conformational folding of the N- and C-terminal protein fusions. All nucleotide sequences encoding the fusion peptide, or other heterologously expressed peptides, can be codon optimized for expression in yeast, and preferably Saccharomyces boulardii.

In another aspect, retinal required for BR function may be produced by the yeast cell, or added exogenously to EVs to reconstitute light-driven proton pumping. In this embodiment, a yeast cell, and preferably a Saccharomyces yeast cell, can be engineered to heterologously express retinal by the co-expression of genes of the beta-carotene biosynthesis pathway including the crtYB, crtI and crtE genes (or from other beta-carotene producing organisms) and the animal beta-carotene cleavage enzyme, beta-carotene 15,15′-dioxygenase which produces trans retinal.

In another preferred aspect, the invention includes novel methods and compositions for loading xenobiotics into EVs produced by yeast. In this embodiment, a target xenobiotic is glycosylated forming a xenobiotic glycoside which can be actively transported across the membrane of the EV into its lumen through a proton driven drug transporter. The glycosidic bond between the xenobiotic and the glycosyl moiety can be hydrolyzed via a glycosidase lumenally anchored to an EV membrane protein.

In another preferred aspect, the invention includes a genetically modified yeast cell configured for the heterologous expression of one or more xenobiotic deconjugating enzymes in a genetically modified yeast cell. In this aspect, a xenobiotic deconjugating enzyme may be expressed in the yeast cell and anchored on the lumenal side of the EV, to an EV membrane protein to regenerate the parent deconjugated compound and to reduce backflow of the conjugate, such as a xenobiotic conjugate, through its membrane transporter.

In another aspect, the EVs of the invention may be modified to display surface ligands that target delivery of the EVs to unique receptors displayed on the surfaces of targeted cell types. The receptor-specific ligands may be fused or anchored to EV membrane proteins and displayed on the EV exterior surface. Examples of such EVs displaying surface ligands for directed transport to target cell receptors are described by Sayre et al., in PCT/US2022/014958, which is incorporated herein by reference.

The present invention includes systems and compositions to genetically modify yeast cells, and preferably a Saccharomyces yeast cell, to express one or more heterologous peptides configured to facilitate the active transport of target compositions, such as preferably therapeutic compositions, into an EV derived from said yeast cell.

In one preferred embodiment, a yeast cell, and preferably a Saccharomyces yeast cell, is engineered to express one or more heterologous fusion proteins including a light-driven proton pump, and a proton driven xenobiotic or xenobiotic conjugate transporter linked to an EV membrane protein. This chimeric protein is configured to orient the fusion peptide of the light-driven proton pump in the EV membrane such that it leads to the light-driven acidification of the EV lumen and the vectorial transport of a target composition, such as xenobiotics or drugs into the EV. The active transport of the target compositions is driven by proton efflux through bacterial and/or eukaryotic small molecule transporters, including xenobiotic transporters.

In another preferred embodiment, a yeast cell, and preferably a Saccharomyces yeast cell, is engineered to express one or more heterologous fusion proteins including a bacteriorhodopsin, and preferably a bacteriorhodopsin according to SEQ ID NO. 2, or a fragment of variation thereof; light-driven proton pump; a proton-driven xenobiotic or xenobiotic conjugate (e.g., glycosides, glucuronic acid, sulfate or glutathione conjugates) transporter linked to an EV membrane protein that is configured to orient the bacteriorhodopsin in the EV membrane, leading to the light-driven acidification of the EV lumen. The vectorial transport of a target composition, such as a xenobiotic or drug, into the EV driven by proton efflux through bacterial and/or eukaryotic small molecule transporters including xenobiotic transporters from the following transporter families: Multidrug and toxic compound extrusion family (MATE); resistance/nodulation/cell division (RND); small multidrug resistance family (SMR); Major facilitator antiporter (MFS); and/or ATP-dependent transporters (ABC).

In another preferred embodiment, the invention is directed to a yeast cell, and preferably a Saccharomyces yeast cell, that is engineered to express one or more heterologous nucleotide sequence, operably linked to a promoter, encoding a fusion construct having:
an EV membrane protein anchor;
a light-dependent proton pump;
optionally a transmembrane domain;
a proton driven drug transporter; and
a linker protein.

In another preferred embodiment, the invention is directed to a yeast cell, and preferably a Saccharomyces yeast cell, that is engineered to express one or more heterologous nucleotide sequence, operably linked to a promoter, encoding a fusion construct having:
an EV membrane protein anchor, and preferably an EV localized Saccharomyces cerevisiae SSO2 or other suitable EV-specific transmembrane protein;
a light-dependent proton pump, and preferably a Haloterrigena turkmenica rhodopsin (BR) or other suitable light-driven proton pumping membrane protein adapted to pump protons into yeast EVs; and
optionally a transmembrane domain, and preferably a human glycophorin A single transmembrane spanning protein, or other odd numbered transmembrane proteins for proper orientation of BR and/or AbeM protein described herein to pump xenobiotics into EVs;
a proton driven drug transporter, and preferably the Acinetobacter baumannii (AbeM) protein, or other suitable proton-driven drug transporters;
a linker protein, and preferably one or more linker proteins, such as (SEQ ID NO 11) adapted to allow proper conformational folding of the N- and C-terminal portions of the fusion peptide.

In one embodiment, the fusion construct of the invention may encode an EV membrane protein anchor comprising a peptide according to the amino acid sequence SEQ ID NO. 1, or a fragment or variant thereof. In a preferred embodiment, the fusion construct of the invention may encode a truncated EV membrane protein anchor fragment comprising a peptide having an amino acid sequence including approximately the first 161 resides of SEQ ID NO. 1, forming a truncated three transmembrane spanning peptide.

In another embodiment, the fusion construct of the invention may encode a light-dependent proton pump comprising a peptide according to the amino acid sequence SEQ ID NO. 2, or a fragment or variant thereof. In another embodiment, the fusion construct of the invention may encode a transmembrane spanning protein according to the amino acid sequence SEQ ID NO. 3, or a fragment or variant thereof. In another embodiment, the fusion construct of the invention may encode a proton driven drug transporter according to the amino acid sequence SEQ ID NO. 4, a fragment or variant thereof. In further embodiments, the heterologous nucleotide construct of the invention is codon optimized for expression in yeast, and preferably Saccharomyces boulardii.

In another preferred embodiment, yeast transgene constructs can be cloned into yeast/E. coli shuttle vectors such as pRS416 and pRS426 for expression of the transgene constructs using the appropriate yeast gene promoter/terminator, such as TDH3p/CYC1t, all of the above being readily known and understood by those skilled in the art.

Retinal required for BR function may be produced by the yeast cell or added exogenously to EVs to reconstitute light-driven proton pumping. In this embodiment, a yeast cell, and preferably a Saccharomyces yeast cell, can be engineering to heterologously express retinal by the co-expression of genes of the beta-carotene synthesis pathway including, the crtYB (SEQ ID NO. 14), crtI (SEQ ID NO. 15) and crtE (SEQ ID NO. 16), or genes from other beta-carotene producing organisms, and the animal beta-carotene cleavage enzyme, beta-carotene 15,15′-dioxygenase which produces trans retinal.

In one aspect of the invention, an exemplary conjugate can include a drug-glycoside. Glycosides are glycoconjugates comprising a sugar covalently attached to another (non-sugar) molecule (the aglycone). Examples in nature include phytochemicals, where the glycoside form is used for storage or transport, and the aglycone is released from the sugar by hydrolysis. Drug glycosides can act as prodrugs, facilitating solubilization and absorption. The prodrugs are then hydrolyzed in the body by glycoside hydrolase enzymes, and the aglycone is then able to perform its therapeutic function. Glycosides can be formed enzymatically by the action of glycosyltransferase enzymes, or by chemical synthesis. Chemical synthesis allows for the glycosylation of a wide variety of API aglycones; enzymatic synthesis is constrained by the substrate specificity of the enzyme. Chemical synthesis via Fischer glycosidation or glycosylation, the Koenigs-Knorr reaction, or chloromethyl synthons are known in the art. Numerous glycoconjugate prodrugs have been described, from daunorubicin (leukemia chemotherapeutic) to propofol (anesthetic) and losartan (treatment of high blood pressure).

In one example, the cancer chemotherapeutic 5-fluorouracil (5-FU) was used as the aglycone. 5-FU is widely used for a number of cancers and is on the List of Essential Medicines of the World Health Organization. It is administered topically and intravenously; both routes lead to side effects, hence the interest in glycosylation of 5-FU for administration as a prodrug. Transport of glyco-5-FU across cell membranes into the cytosol is mediated by sodium dependent glucose transporter 1 (SGLT1), which normally occurs in the mammalian small intestine; it has been functionally expressed in the yeast Saccharomyces cerevisiae, where it displayed both influx and efflux of substrate, depending on the orientation of the substrate concentration gradient. Hydrolysis of glyco-5-FU has been demonstrated using beta-glucosidase from Agrobacterium sp. (ABG; EC 3.2.1.21, CAZy GH1), a protein of 459 residues (52.2 kDa).

Co-expression of a xenobiotic deconjugating enzyme in yeast EVs linked to the lumenal side of an EV membrane allows the system to regenerate the parent xenobiotic and to reduce loss of xenobiotic conjugates (glycosides, glutathione conjugates, etc.) from the EVs and to make the xenobiotic biologically more active than its conjugate. In this example, yeast (Saccharomyces boulardi) was engineered to express sodium dependent glucose transporter 1 (SGLT1) (SEQ ID NO. 13), and beta-glucosidase (ABG) (SEQ ID NO. 12), and to target them to EVs by tethering them genetically to domains of EV-resident proteins. The chemotherapeutic agent 5-fluorouracil (5-FU) was chemically glycosylated. The purified glyco-5-FU was then administered to the engineered EVs. Transport of glyco-5-FU into the EVs via SGLT1, and its hydrolysis to 5-FU and glucose by ABG, were further demonstrated.

In another embodiment, the invention is directed to genetically modified yeast cells adapted to heterologously express and present on the surface of EVs receptor-specific ligands anchored to EV membrane proteins as a fusion peptide. In a preferred embodiment, the anchor portion of the fusion peptide can include one of several EV membrane localized proteins including, Chr1 (GPI-anchored cell wall protein) (SEQ ID NO. 5), Sur7 (multi-pass membrane protein) (SEQ ID NO. 6) or Nce102 (non-classical export protein) (SEQ ID NO. 7), or fragments or variants thereof. In another embodiment, cancer cell specific or enriched receptor ligands can be anchored to the EV membrane protein C- or N-terminus so that the ligands are displayed on the surface of the EV.

More specifically, in one embodiment surface displayed ligands to target EVs to P-selectin receptors, which are over-expressed in blood vessels of a variety of cancers, can be selected. Specific embodiments may include one or more of the following: NRP1 receptor, follicle-stimulating hormone (FSH) receptor FSHR; epidermal growth factor (EGF) receptor. The examples of receptor-binding ligands used for fusion are C-end class peptides for binding NRP1 receptor (SEQ ID NO. 8); AEYLR-small peptide binding EGFR (SEQ ID NO. 9); small peptide FSH33-53 for binding FSHR (SEQ ID NO. 10).

In one embodiment, one or more of the EVs of the invention may include a pharmaceutical composition. A “pharmaceutical composition” of the invention includes a composition of the invention and a pharmaceutically acceptable carrier. The term “pharmaceutically acceptable” as used herein, refers to molecular entities and compositions that do not produce adverse, allergic, or other untoward reactions when administered to an animal or a human. The term, “pharmaceutically acceptable carrier” as used herein, includes any and all solvents, or a dispersion medium including, but not limited to, water, ethanol, a polyol (for example, glycerol, propylene glycol, and liquid polyethylene glycol, stachyose, and the like), suitable mixtures thereof, and vegetable oils, coatings, isotonic and absorption delaying agents, liposomes, commercially available cleansers, and the like. Supplementary bioactive ingredients also can be incorporated into such carriers.

As used herein “xenobiotic” refers to a chemical or other compound, whether biological or non-biological which is not a normal component of an organism which is exposed to. Exemplary xenobiotic compositions include, tkanamycin, gentamicin, ofloxacin, ciprofloxacin, erythromycin, chloramphenicol, DAPI, triclosan, acriflavine, Hoechst 33342, daunomycin, doxorubicin, trimethoprim, and/or rhodamine 6G among others.

The term “endogenous” gene or protein means that said gene or protein is expressed from a gene naturally found in the genome of a eukaryotic cell. The term “heterologous” gene or protein means that said gene or protein is not expressed from a gene naturally found in the genome of a eukaryotic cell. As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the phrase “expression,” “gene expression” or “protein expression,” such as the level of includes any information pertaining to the amount of gene transcript or protein present in a sample, in a cell, in a patient, secreted in a sample, and secreted from a cell as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information.” Such information may be given in the form of amounts per cell, amounts relative to a control gene or protein, in unitless measures, etc.

The term “fusion protein” as used herein is used as it is in the art. Namely, the fusion proteins used in the methods and compositions of the present invention involve two separate proteins or protein domains that are linked by a covalent bond. In one embodiment, the covalent bond linking the two domains is an amine bond. In more specific embodiments, the anchor protein and the foreign protein is a fusion protein comprising a single-chain polypeptide. In even more specific embodiments, the single-chain polypeptide comprising the anchor protein and the foreign protein further comprises a linker peptide sequence. Any linker sequence can be used to covalently link the anchor protein and the foreign protein.

As used herein, the term “peptide linker(s),” “linker(s),” or “linker moiety” refers to a peptide or polypeptide sequence, e.g., a synthetic peptide or polypeptide sequence, which connects two domains in a linear amino acid sequence of a polypeptide chain. In one embodiment, the polypeptides of invention are encoded by nucleic acid molecules that encode peptide linkers which either directly or indirectly connect the anchor protein and foreign protein which make up the construct. These linkers may be interposed between the anchor protein and foreign protein. If the linker connects two protein moieties contiguously in the linear polypeptide sequence, it is referred to as a “direct” linkage. In contrast, the linkers may link the first protein moiety, i.e., anchor protein or foreign protein, to a binding moiety which is, in turn, linked to the second protein moiety, i.e., anchor protein or foreign protein, thereby forming an indirect linkage. Linkers are typically located at the N or C terminus of the protein moieties.

In one embodiment, the linker linking the anchor protein and the foreign protein is a peptide comprised of glycine (Gly) n, wherein n is an integer that is the same or higher than 1, 2, 3, 4, 5, 6, 7, 8, 9, 10. In another embodiment, the linker linking the anchor protein and the foreign protein is a gly-ser linker. As used herein, the term “gly-ser peptide linker” (GS) refers to a peptide comprising or consisting of glycine (G or Gly) and serine (Sor Ser) residues. Exemplary gly-ser peptide linkers comprise the amino acid sequence (Gly4 Ser) n or (Gly3 Ser) n. Another exemplary gly-ser peptide linker comprises the amino acid sequence S (Gly4 Ser) n wherein n is an integer that is the same or higher than 1, 2, 3, 4, 5, 6, 7, 8, 9, 10. In another embodiment, the linker linking the anchor protein and the foreign protein is a peptide comprising SEQ ID NO. 11.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. A “variant” includes a peptide having the same function as an identified peptide, while having a different sequence or being derived from a different organism than an identified peptide.

As used herein, a “fragment” of poly- or oligonucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g., 5, 6, 7, 8, 9, 10, 11 or 12 or more amino acids long, including each integer up to the full length of the polypeptide). Those skilled in the art are well versed in methods for selecting antigenic fragments of proteins.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “introducing,” “administered” or “administering”, as used herein, refers to any method of providing a composition of EVs to a patient such that the composition has its intended effect on the patient. In one embodiment, EVs may be introduced to a patient in vivo, while in other alternative embodiments, EVs may be introduced to subject cells in vitro which may then be administered to a patient in vivo.

The term “patient,” or “subject” as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connotes a need for medical treatment, therefore, a patient may voluntarily or involuntarily be part of experimentation whether clinical or in support of basic science studies.

Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “expression cassette” refers to a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The peptides of the invention of the present invention may be chimeric.

The expression cassette may also be one which is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter which initiates transcription only when the host cell is exposed to some particular external stimulus.

As used herein, a promoter region or promoter element refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

To accomplish delivery of therapeutic compositions, such as small-molecule drugs, biologics, or other xenobiotics to target cells, the methods and compositions of the present invention comprise extracellular vesicles (EVs), and preferably EVs generated from Saccharomyces, such as S. boulardii or S. cerevisiae. EVs generated from Saccharomyces sometimes are also interchangeably referred to as Saccharomyces-generated extracellular vesicles, sometimes also referred to as SGEVs. The term extracellular vesicles refer to membranous vesicles released from cells. The extracellular vesicles of the methods and compositions of the invention are composed of lipid bilayers that can be loaded with and carry cargo in its interior. The lipid bilayer of the EVs may also include proteins embedded therein. In some embodiments, the SGEVs of the compositions and methods of the present invention can be exosomes or ectosomes. As is well-known, exosomes are generally formed upon the endocytosis of multivesicular endosomes (MVEs) to form intraluminal vesicles (ILVs) which are subsequently released into the extracellular environment as exosomes, whereas ectosomes are assembled at and released from the plasma membrane. Often, the primary structural feature distinguishing ectosomes and ectosomes is diameter. In some embodiments, the diameter of the SGEVs are between about 30 nm to about 180 nm, between about 50 nm to about 200 nm, between about 75 nm to about 250 nm, between about 100 nm to about 300 nm, between about 125 nm to about 350 nm, between about 150 nm to about 400 nm, between about 175 nm to about 450 nm, between about 200 nm to about 500 nm, between about 250 nm to about 550 nm, between about 300 nm to about 600 nm, between about 350 nm to about between about 650 nm, between about 400 nm to about 700 nm, between about 450 nm to about 750 nm, between about 500 to about 800 nm, between about 550 nm to about 850 nm, between about 600 nm to about 900 nm, between about 650 nm to about 950 nm, between about 700 nm to about 1000 nm, between about 750 nm to about 1050 nm, between about 800 nm to about 1100 nm, between about 850 nm to about 1150 nm or between about 900 nm to about 1200 nm. Thus, exosomes may comprise components on their membrane surface, including but not limited to, proteins, glycoproteins, proteoglycans, carbohydrates, and lipids, which may be used to direct delivery of therapeutic compositions loaded into EVs.

As understood by the disclosure herein, Saccharomyces is a single-celled organism, but the term “extracellular vesicle,” as it relates to the SGEVs, refers to vesicles that are secreted from Saccharomyces into the local environment, such as, but not limited to, cell culture medium or the gastro-intestinal tract of organisms that may have ingested or consumed or been administered the Saccharomyces, which secrete the vesicles containing a therapeutic composition. In one embodiment, the SGEVs are secreted from Saccharomyces cerevisiae or Saccharomyces boulardii.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the peptides may be identical to the coding sequence shown in the sequence listing, or that of any of the deposited clones, or may be a different coding sequence which, as a result of the redundancy or degeneracy of the genetic code, encodes the same fusion proteins as shown in the sequence listing.

The term “nucleotide sequence encoding a peptide” encompasses a nucleotide sequence which includes only coding sequences for the polypeptide, e.g., heterologous protein, as well as a polynucleotide which includes additional coding and/or non-coding sequences. Thus, for example, the polynucleotides of the present invention may encode for a peptide, e.g., a heterologous protein, or for a peptide having a prosequence or for a protein having both a prosequence and presequence.

The polynucleotides of the present invention may also have the coding sequence fused in frame to, for example, a marker sequence which allows for identification of the polypeptide of the present invention. The marker sequence may be a GFP protein, a hexa-histidine tag to provide for purification of the fusion protein is used.

The invention also relates to vectors, including but not limited to, expression vectors comprising the polynucleotides encoding the fusion proteins of the present invention. Types of vectors for expression for proteins and fusion proteins are well known in the art. In one embodiment, the vector is an expression vector for protein expression in Saccharomyces. Yeast expression vectors are commercially available from manufacturers.

The present invention also relates to methods of making and using these Saccharomyces-generated EVs. In one embodiment, the methods of making the SGEVs of the present invention comprise introducing into the Saccharomyces the expression vector encoding one or more heterologous proteins and related systems which actively transport therapeutic compositions into the EV, or a fragment or variant thereof, of the present invention to generate a host Saccharomyces cell. The host cell is then cultured under conditions to permit protein production from the vector encoding the heterologous protein. In one embodiment, the host cells of the present invention are Saccharomyces cerevisiae or Saccharomyces boulardii.

Culture conditions for culturing yeast host cells are well-known in the art. The continued culture of the host cell will permit production and secretion of the SGEVs into the cell culture environment, where they can be isolated from culture. Methods of isolating extracellular vesicles, such as exosomes, from cell culture media are well-known in the art and are reviewed in Li, P. et al., Theranostics, 7(3): 789-804 (2017), which is incorporated by reference herein. Methods of isolating the SGEVs from culture include but are not limited to ultracentrifugation methods, size-based enrichment methods (e.g., tangential flow filtration, size-exclusion chromatography), immunoaffinity capture-based methods, precipitation methods, microfluidics-based methods or some combination thereof.

The route of administration of the SGEVs includes, but is not limited to, topical, transdermal, intranasal, rectal, oral, subcutaneous, intravenous, intraarterial, intramuscular, intraosseous, intraperitoneal, epidural and intrathecal as disclosed herein. In one example SGEV's may be derived or isolated from a GRAS and/or probiotic yeast cell, such as Saccharomyces cerevisiae, and preferably Saccharomyces boulardii. For example, Saccharomyces boulardii probiotics, releasing wild type exosomes, have been shown to diminish disease severity by reducing the expression of inflammatory cytokines and stimulating the expression of anti-inflammatory cytokines in multiple organs including the lungs and cardiovascular system. Saccharomyces boulardii cells also have low immunogenicity and positively modulate host immune response in the presence of additional antigens. S. boulardii is well established for allowing the present inventors to engineer the S. boulardii strain for expression and loading of specific xenobiotics in exosomes. Cultivation of S. boulardii is fast, low-cost, and easy to scale up using established procedures.

Finally, the lipids present in EVs are natural and thus not likely to be cytotoxic when used therapeutically unlike artificial lipids frequently used to package mRNA for vaccines. In specific embodiments, the oral administration of the SGEVs includes administering engineered yeast, producing the SGEVs, as a probiotic. As used herein, a probiotic is a microorganism, such as a bacteria or yeast, generally recognized as safe for human or animal consumption. The probiotics of the present invention may or may not have additional health benefits to the consumer. In specific embodiments of the present invention, the probiotic is a Saccharomyces cerevisiae or a Saccharomyces boulardii.

The terms “comprises”, “comprising”, are intended to have the broad meaning ascribed to them in U.S. Patent Law and can mean “includes”, “including” and the like.

The invention now being generally described will be more readily understood by reference to the following examples, which are included merely for the purposes of illustration of certain embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Overview and Experimental Rationale

S. boulardii produces extracellular vesicles (sometimes referred to herein as yEV or EV) that may package RNA, proteins, and other small molecules. S. boulardii can also be genetically engineered for the purpose of modifying yEV targeting or cargo. To develop a process for actively loading therapeutic small molecular weight drugs into yeast extracellular vesicles Applicants have incorporated into the yEV membrane a multidrug/proton antiporter, AbeM fused to the C-terminus of truncated yEV membrane protein Sur7. AbeM is a multidrug transporter of the acid-loving bacterium Acinetobacter baumannii and belongs to the MATE (multidrug and toxic efflux) class of multidrug transporters which function also as proton antiporters. In the presence of a transmembrane proton gradient, AbeM has been demonstrated to transport multiple drugs including, rhodamine G, ethidium bromide, various antibiotics, and doxorubicin. Doxorubicin was used as a model drug because of its anti-cancer properties and the fact that it is highly solubility in aqueous buffers and fluorescent.

Applicants designed protein encoding gene constructs that allow the expression of AbeM in two possible membrane orientations-inward and outward pumping when fused to the Sur7 yEV membrane anchoring protein. To generate an inward drug transport orientation, the AbeM gene was linked to a truncated Sur7 protein (SEQ ID NO. 17) containing only the first 3 transmembrane helixes. This gene construct name is identified as SEQ ID NO. 19, and is further abbreviated as “Tr”. To achieve an outward drug pumping orientation, AbeM was fused to the C-terminus of the full-length Sur7 gene (SEQ ID NO. 19), and this construct is identified as SEQ ID NO. 18, and is further is abbreviated as “Full.”

Notably, only one of the orientations will transport drugs into yEVs when driven by a proton gradient across the membrane. According to the AbeM crystal structure, the N- and C-terminus of the AbeM protein are located on the cytoplasmic (high pH side) surface of the bacterial membrane. The AbeM orientation predicted in the truncated Sur7 gene fusion with AbeM would result in an AbeM orientation that would pump drugs from the outside of the yEV (high pH) to the inside (low pH) of the yEV.

A third AbeM construct (SEQ ID NO. 21) was also designed lacking the N-terminal fusion to the yEV membrane protein Sur7. In this construct AbeM protein sequence includes the first 29 amino acids of the bacterial AbeM signal motif for localization in the bacterial membrane. However, according to ProtComp 9, a program used for the identification of sub-cellular localization of eukaryotic proteins (Animal/Fungi) AbeM contains a putative fungal signaling sequence (aa 31-50) for localization in the yeast plasma membrane and a potential GPI-anchor. Positioned at the C-terminus the putative glycosylphosphatidylinositol (GPI) anchor is a posttranslational modification that anchors the modified protein in the outer leaflet of the cell membrane. To ensure localization of AbeM in yEV membrane, a protein yEV sorting motif of 59 amino acids (SEQ ID NO. 22) obtained from the C-terminus of chitinase (Uniprot number A0A0L8VL70) was included at the C-terminus of the AbeM-p-ESM construct to also localize the AbeM protein in the yEV membrane.

### Example 2: Materials and Methods

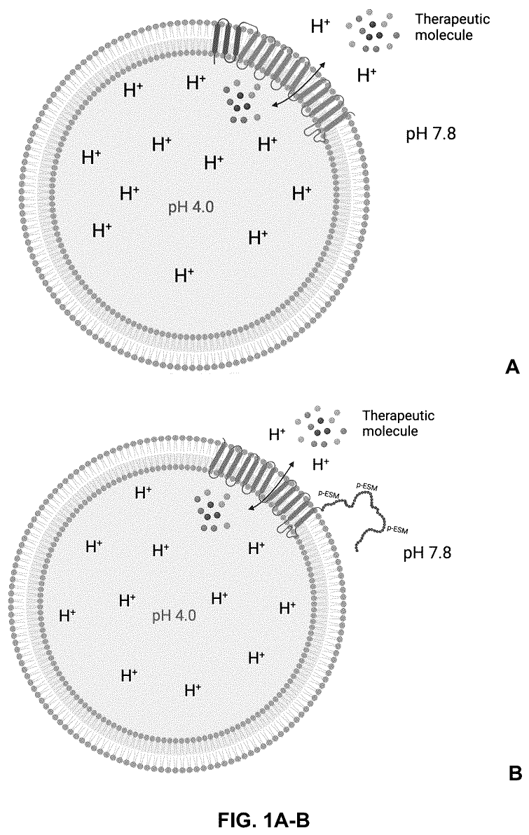

S. boulardii strain design and construction: To create S. boulardii strains expressing the AbeM protein fused with a full or truncated version of SUR7 gene, the wild-type S. boulardii was transformed with linear dsDNA segments including the Sur7-AbeM expressing cassette consisting of a TDH3 gene promoter, genes of interest, aCYC1 terminator and geneticin-resistance gene flanked on 5′ and 3′ ends by integration sequences homologous to sequences from YPRCt3 locus on XVI chromosome. The dsRNA fragments were synthesized by Genscript. Yeast transformation was performed by electroporation following the protocol described by Benatuil et al (2010). A yEV membrane protein model of the truncated Sur7-AbeM fusion and pH driven drug transport is shown in FIG. 1.

yEV isolation: Overnight cultures of S. boulardii were diluted 100 times with YPD medium. Cultures were then incubated for 24 h at 30° C. with shaking (200 rpm). For yEV isolation, cells and debris were removed by centrifugation at 3500×g for 35 min. yEVs were then concentrated from the supernatant using a tangential flow filtration device (Pall) with 300 kD membrane to a yEV concentration approximately 5×10¹¹ yEVs/mL. Isolated yEVs were aliquoted and stored in PBS buffer at −80° C.

yEV quantification: The concentration of yEVs was measured by Nanoparticle Tracking Analysis (NTA) using a Particle Metrix NTA. All samples were diluted in water to a final volume of 1.5 mL. Proper measurement concentrations were obtained by diluting samples until a concentration of 140-200 particles/frame was achieved. For each measurement, three cycles of measurements were performed by scanning 11 positions, each cycle was carried out under the following settings: Focus: autofocus; Camera sensitivity for all samples: 75; Shutter: 80; Scattering Intensity: 30; Cell temperature: 25° C. To obtain the number of yEVs per mL the video recordings were analyzed by the equipment software.

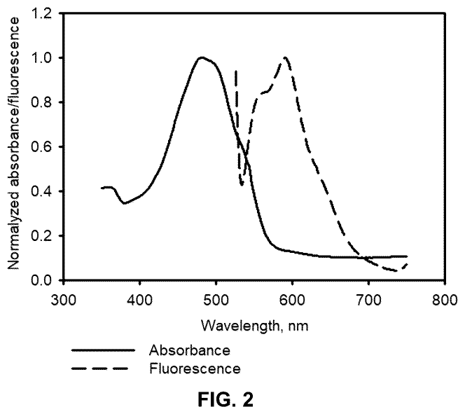

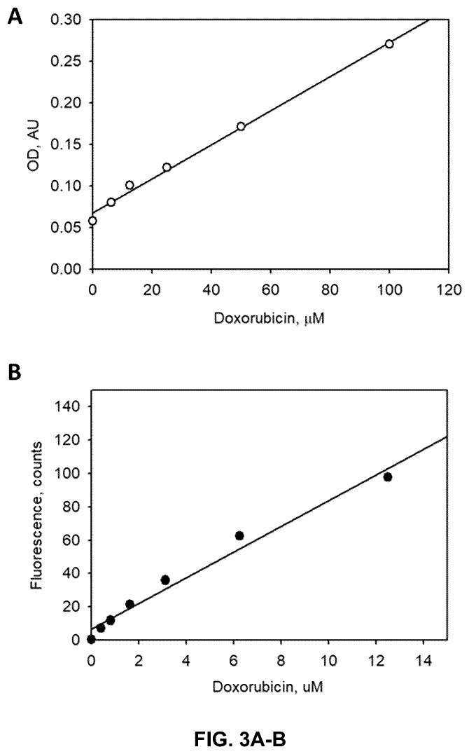

yEVs electroporation: A 10 mM stock solution of doxorubicin was mixed with yEVs diluted in PBS to a final concentration of 2 mM doxorubicin and 2×10¹¹ yEVs/mL. The yEV-doxorubicin mixture was incubated for 30′ at 4° C. and then mixed with the electroporation buffer (400 mM sucrose in PBS) at a 1:1 ratio. 2 mL of the sample was electroporated in a 0.4 cm cuvette by exponential pulse with set at 950 V, 50 uF using a GenePulser Xcell; (BioRad) electroporation system. The electroporated yEVs were then incubated for 30′ at 37° C. and free doxorubicin was removed from the yEV sample by separate 20 mL washes using sequentially 100 mM sucrose and 50 mM sucrose in PBS, followed by 2 repeat washes with PBS using a 300 kD cut off filter to concentrate the yEVs (Sartorius). The final volume of the sample was adjusted to 1 mL using the centrifugal filtration unit. The doxorubicin fluorescence was measured using a spectrophotometer plate-reader SpectraMax M4 (Molecule Devices) at 470 nm excitation/560 nm emission settings and the amount of doxorubicin was calculated by fitting the experimental result to a calibration curve of the doxorubicin solution FIG. 2.

Human cell culture: Human lung cells (H1299 cells) (ATCC CRL-5803) were maintained in RPMI-1640 medium supplemented with 10% fetal bovine serum (FBS; Gibco, Carlsbad, CA; #26140) and 100 U/mL penicillin/streptomycin (Invitrogen, Carlsbad, CA; #15140) and grown at 37° C. and 5% CO₂.

Human cells visualization and doxorubicin imaging: For live cell imaging for doxorubicin uptake, either free doxorubicin or doxorubicin loaded into yEVs was added to H1299 cells plated on a glass-bottomed dish. Images were taken using an inverted fluorescent microscope (VWR 89404-464) using 20× or 40× objectives. For visualization of cell nucleus, Hoechst dye 33342 was added to the medium in final concentration 2 μM. Images were processed using ImageJ (1.47v) software.

Buffer exchange of yEV preps: Both fast buffer exchange of EVs preps and cleanup of unincorporated doxorubicin from yEV preps was performed by column gel-filtration using PCR Kleen™ Spin Columns (Bio-Rad, 7326300). 50-100 μL of yEVs suspension was applied to the center of the column prepared by manufacturer recommendations. Columns were immediately spun at 800×g for 2 min. Samples were collected at the bottom of 1.5 mL centrifuge tubes.

Doxorubicin quantification using plate reader: The quantum yield of doxorubicin in a water solution is about ˜0.05 (FIG. 2). Absorbance and fluorescence emission spectra were taken using SpectraMax, M4 plate-reader (Molecular Devices) (Shah et al., 2017)

### Example 3: Loading of Doxorubicin into yEVs by Electroporation

Electroporation for the loading of yEVs with doxorubicin was repeated four times with average loading yield 0.79 μg per 10¹⁰ yEVs (SE=0.3). The total amount of recovered yEVs was measured on ZetaView NTA, and the drug loading efficiency was determined by fluorescence signal measurements at 470/560 nm Ex/Em on a SpectraMax M4.

### Example 4: Cellular Uptake of Free Doxorubicin and Doxorubicin Loaded into yEVs

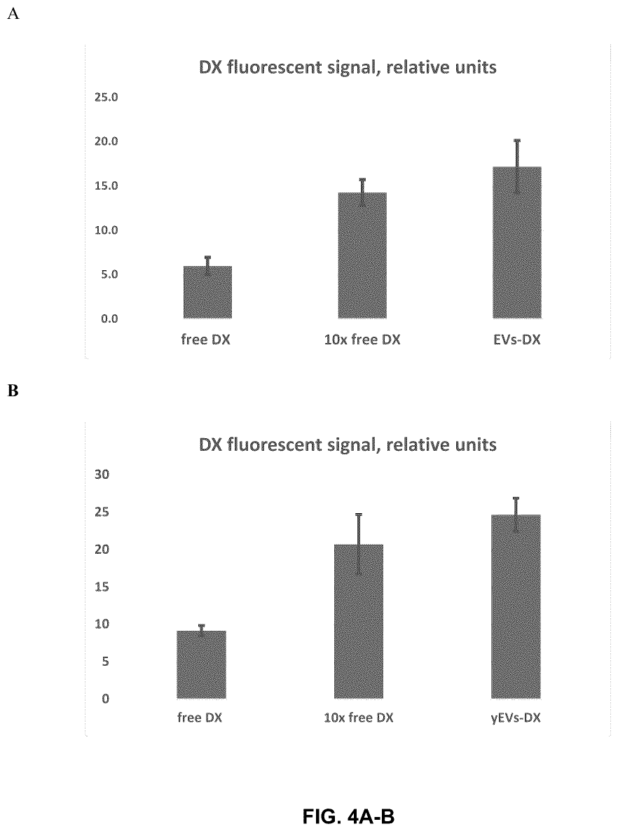

Free doxorubicin at concentrations of 0.45 μg/ml or 4.5 μg/mL and of doxorubicin (0.45 μg/mL) loaded into yEVs was incubated with H1299 human lung cells for 2 hours at 37 C. Cells were then rinsed 3 times with fresh RPMI media to remove non-incorporated doxorubicin and images of cells were taken using an RFP filter set (excitation 545-580 nm, emission 610) using the same exposure settings for all samples. The fluorescent signal attributed to doxorubicin captured by cells was measured with ImageJ to compare the efficiency of doxorubicin uptake. Applicants observed that more than a 10-fold greater concentration of free doxorubicin was required to yield the same doxorubicin fluorescence signal intensity compared to doxorubicin delivered by yEVs (FIG. 4)

### Example 5: Proton Gradient Formation

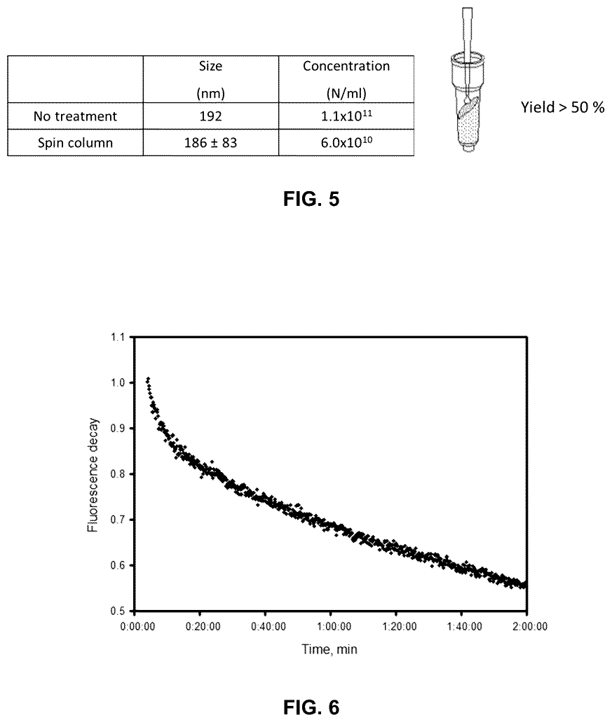

To load doxorubicin into AbeM-expressing EVs, Applicants ensured the formation of the proton gradient across the yEV membrane. Applicants equilibrated yEVs with protons by overnight incubation with acid buffers. To preserve AbeM integrity a buffer pH equal to 3 or higher was used to acidify the yEV lumen. To create a yEV transmembrane pH gradient, Applicants did a very fast buffer exchange by spin-column gel filtration. As shown in figure below (FIG. 5) it is apparent that this acidification treatment had no apparent impact on yEV particle size integrity or yield.

To make sure that a proton gradient was generated, Applicants monitored proton release using the fluorescent pH indicator Oregon Green. At neutral pH, Oregon Green has high fluorescence which decreases at acid pH. To monitor the transmembrane fluorescence change, 75 μL of 50 nM of OG solution in 50 mM Tris-HCl pH 8.0 was mixed with 25 μl freshly column-exchanged proton-loaded yEVs in the well of a black microplate. Changes in fluorescence in response to proton release from acidified yEVs were monitored by fluorescence (λex 485 nm, cutoff 495 nm, λem 525 nm) using the kinetic mode of plate reader Spectramax M4. As shown in FIG. 6, the pH gradient degrades slowly (>2 h). Thus, Applicants incubated acid-equilibrated yEVs for a longer time, ˜16 h.

### Example 6: Doxorubicin Uptake into Wild-Type and Sur7-AbeM-Expressing yEVs

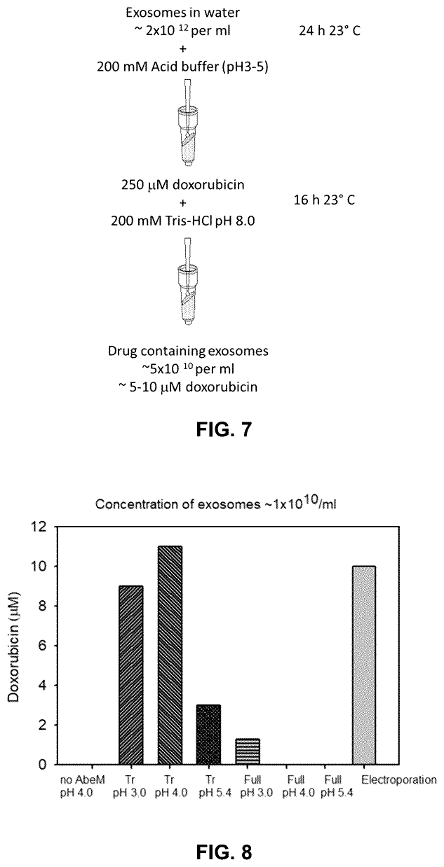

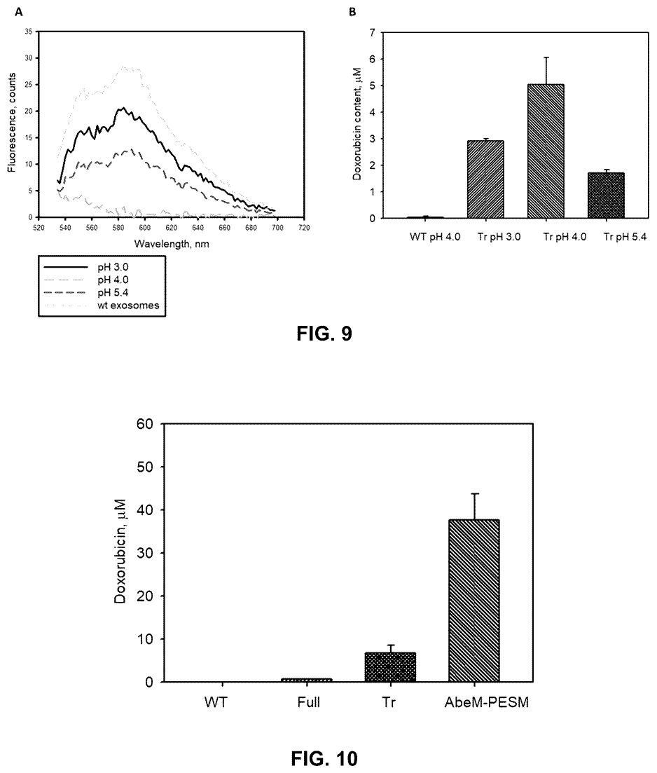

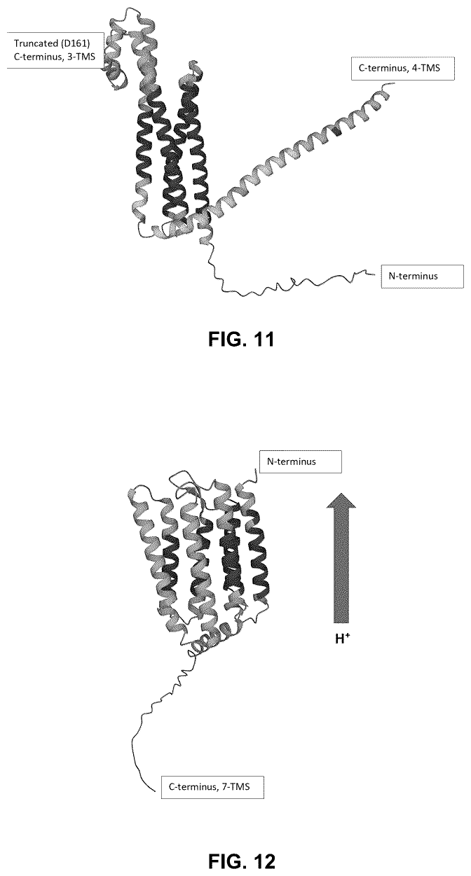

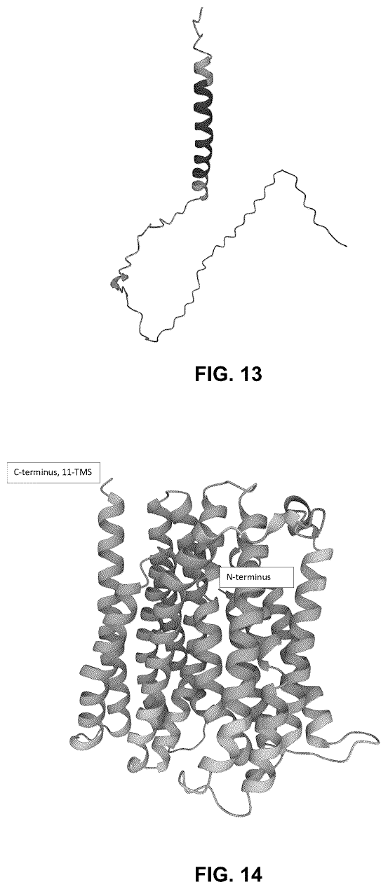

yEVs preps were prepared as described above as a water-suspension. The general scheme of procedure is presented in FIG. 7. To create pH gradients, yEVs were equilibrated in buffers of acid pH overnight at room temperature. The following buffers were used for the acid equilibration of yEVs: 100 mM Glycine pH 3.0, 100 mM MES pH 4.0, and 100-mM potassium acetate, pH 5.4. After incubation samples were quickly transferred to 10 mM Tris pH 7.5 and immediately mixed with equal amounts of 400 mM Tris pH 8.0, 500 μM doxorubicin. Following incubation for 16 h at room temperature unincorporated doxorubicin was removed by gel-filtration as described above. yEV preps were analyzed on doxorubicin content, aliquoted, and stored at −80° C.

### Example 7: Proper AbeM Orientation in the Membrane for Drug Pumping into yEVs

To determine the working orientations of AbeM, Applicants used preps of yEVs in which AbeM was linked to the C-terminus of either a truncated (Tr) and full (Full) Sur7 protein that serves as an anchor for localization in the yEV membrane. Doxorubicin content was detected by fluorescence and concentrations were determined using the calibration curve. In this experiment, Applicants found that doxorubicin uptake was observed only in Tr constructs. No significant uptake of doxorubicin was observed in yEVs expressing the Full Sur7-AbeM protein fusion or in yEVs without AbeM. Thus, all future experiments were performed with the Tr construct.

### Example 8: Optimal pH for Doxorubicin Uptake

To determine optimal conditions for doxorubicin uptake into Tr-yEVs, Applicants compared doxorubicin uptake using yEVs equilibrated at various acid pHs followed by transfer to approximately pH 8 media plus doxorubicin (500 μM). We observed that a yEV lumenal pH of approximately 4.0 was optimal for doxorubicin uptake.

### Example 9: Doxorubicin Uptake by yEVs Expressing the AbeM-p-ESM Construct

Using the established pH gradient driven drug loading conditions, Applicants analyzed doxorubicin uptake into yEVs containing the AbeM-p-ESM protein. AbeM-p-ESM-yEVs were equilibrated at pH 4.0 for 16 h in MES pH 4. Using a spin column, buffer was exchanged into 100 mM Tris-HCl pH 8.0 supplemented with 250 μM doxorubicin. After incubation for 24 h, excess of doxorubicin was removed, and doxorubicin content was analyzed by fluorescence emission using a plate reader. As shown in FIG. 10, AbeM-p-ESM-yEVs had 6-fold greater drug uptake than Tr-yEV and 50-fold greater doxorubicin uptake than Full-yEVs construct. Doxorubicin-loaded AbeM-PESM-yEVs are visibly colored and may easily detected by both spectrophotometry and fluorescence. Drug loading on AbeM-p-ESM-yEVs was also much faster. Applicants found that 50% of doxorubicin was loaded after 1 h incubation with drug. Each AbeM-p-ESM-yEV is estimated to contain 5-10 molecules of doxorubicin.

### Example 10: Results Summary

Applicants demonstrate that yEVs expressing AbeM linked to the truncated Sur7 yEV membrane protein having three transmembrane spanning domains supports significant uptake of doxorubicin when the internal pH of the yEV lumen is approximately pH 4 and the external approximately pH is 8, unlike yEVs expressing AbeM linked to the Full Sur7 yEV membrane protein having 4 transmembrane spanning domains.

Applicants describe conditions for generating a pH gradient for loading doxorubicin loading into yEVs expressing the truncated Sur7-AbeM construct. We note that pH 4 is more optimal than pH 3 or pH 5 in certain embodiments, however in specific other embodiments optimal pH can differ.

AbeM-mediated and proton dependent drug uptake can be an effective means to deliver drugs such as doxorubicin by yEVs to live cells than by using free drugs in solution substantially reducing the potential for off-target side effects.

Unlike electroporation mediated drug uptake into yEVs, AbeM-p-ESM mediated drug loading into yEVs is industrially scalable by pre-equilibrating a large volume of yEVs at pH 4.0 and then transferring the yEVs to pH 7.8 plus drug for 1 hour.

The ability to efficiently deliver hydrophilic and/or hydrophobic drugs in yEVs in a targeted manner to specific organs and cells substantially reduces the potential for off-target side effects and can effectively lower the drug dosage required for effective disease management.

### REFERENCES

1. Abdi S N, Ghotaslou R, Ganbarov K, Mobed A, Tanomand A, Yousefi M, et al. Acinetobacter baumannii efflux pumps and antibiotic resistance. Infect Drug Resist. 2020; 13:423-34.
2. Bukowski K, Kciuk M, Kontek R. Mechanisms of multidrug resistance in cancer chemotherapy. Int J Mol Sci. 2020; 21(9).
3. Dawson C S, Garcia-Ceron D, Rajapaksha H, Faou P, Bleackley M R, Anderson M A. Protein markers for Candida albicans EVs include claudin-like Sur7 family proteins. J Extracell Vesicles [Internet]. 2020; 9(1). Available from:
4. Delmar J A, Su C C, Yu E W. Bacterial multidrug efflux transporters. Annu Rev Biophys. 2014; 43(1):93-117.
5. Díez-Sampedro A, Lostao M P, Wright E M, Hirayama B A. Glycoside binding and translocation in Na (+)-dependent glucose cotransporters: comparison of SGLT1 and SGLT3. J Membr Biol. 2000 Jul. 15; 176(2):111-7. doi: 10.1007/s00232001081.
6. Du D, van Veen H W, Murakami S, Pos K M, Luisi B F. Structure, mechanism and cooperation of bacterial multidrug transporters. Curr Opin Struct Biol 2015; 33(FIG. 1):76-91. Available from:
7. Du D, Wang-Kan X, Neuberger A, van Veen H W, Pos K M, Piddock L J V, et al. Multidrug efflux pumps: structure, function and regulation. Nat Rev Microbiol [Internet]. 2018; 16(9):523-39. Available from:
8. Fu D, Bebawy M, Kable E P W, Roufogalis B D. Dynamic and intracellular trafficking of P-glycoprotein-EGFP fusion protein: Implications in multidrug resistance in cancer. Int J Cancer. 2004; 109(2):174-81.
9. Elferink H, W. H. C. Titulaer, M. G. N. Derks, G. H. Veeneman, F. P. J. T. Rutjes, T. J. Boltje, Chem. Eur. J. 2022, 28, e202103910.
10. Firnges M A, Lin J T, Kinne R K. Functional asymmetry of the sodium-D-glucose cotransporter expressed in yeast secretory vesicles. J Membr Biol. 2001 Jan. 15; 179(2):143-53. doi: 10.1007/s002320010044.
11. Fu D, Bebawy M, Kable E P W, Roufogalis B D. Dynamic and intracellular trafficking of P-glycoprotein-EGFP fusion protein: Implications in multidrug resistance in cancer. Int J Cancer. 2004; 109(2):174-81.
12. Han C Y, Yue L L, Tai L Y, Zhou L, Li X Y, Xing G H, Yang X G, Sun M S, Pan W S. A novel small peptide as an epidermal growth factor receptor targeting ligand for nanodelivery in vitro. Int J Nanomedicine. 2013; 8:1541-9. doi: 10.2147/IJN.S43627. Epub 2013 Apr. 19. PMID: 23626467; PMCID: PMC3632632.
13. Haspel N, Zanuy D, Nussinov R, Teesalu T, Ruoslahti E, Aleman C. Binding of a C-end rule peptide to the neuropilin-1 receptor: a molecular modeling approach. Biochemistry. 2011 Mar. 15; 50 (10):1755-62. doi: 10.1021/bi101662j. Epub 2011 Feb. 14. PMID: 21247217; PMCID: PMC3051018.
14. He G X, Kuroda T, Mima T, Morita Y, Mizushima T, Tsuchiya T. An H+-Coupled Multidrug Efflux Pump, PmpM, a Member of the MATE Family of Transporters, from Pseudomonas aeruginosa. J Bacteriol. 2004; 186(1):262-5.
15. Jornada D H, dos Santos Fernandes G F, Chiba D E, de Melo T R, dos Santos J L, Chung M C. The Prodrug Approach: A Successful Tool for Improving Drug Solubility. Molecules. 2015 Dec. 29; 21(1):42. doi: 10.3390/molecules21010042
16. Kapoor V, Wendell D. Engineering bacterial efflux pumps for solar-powered bioremediation of surface waters. Nano Lett. 2013; 13(5):2189-93.
17. K. Kassiani, A. Marta, H. Overkleeft, J. Aerts. Plant Glycosides and Glycosidases: A Treasure-Trove for Therapeutics Front. Plant Sci. 2020 11:10.3389/fpls.2020.00357
18. K L R. Integration of hepatic drug transporters and phase II metabolizing enzymes: Mechanisms of hepatic excretion of sulfate, glucuronide, and glutathione metabolites. Eur J Pharm Sci. 2006; 27(5):447-86.
19. Kusakizako T, Miyauchi H, Ishitani R, Nureki O. BBA—Biomembranes Structural biology of the multidrug and toxic compound extrusion superfamily transporters. BBA-Biomembranes 2020; 1862(12):183154.
20. Lairson L L, Henrissat B, Davies G J, Withers S G. Glycosyltransferases: structures, functions, and mechanisms. Annu Rev Biochem. 2008; 77:521-55. doi: 10.1146/annurev.biochem.76.061005.092322
21. Oliveira D L, Nakayasu E S, Joffe L S, Guimarães A J, Sobreira T J P, Nosanchuk J D, et al. Characterization of yeast extracellular vesicles: Evidence for the participation of different pathways of cellular traffic in vesicle biogenesis. PLOS One. 2010; 5(6):1-13.
22. Omote H, Hiasa M, Matsumoto T, Otsuka M, Moriyama Y. The MATE proteins as fundamental transporters of metabolic and xenobiotic organic cations. Trends Pharmacol Sci. 2006; 27(11):587-93.
23. Pos K M. Drug transport mechanism of the AcrB efflux pump. Biochim Biophys Acta-Proteins Proteomics 2009; 1794(5):782-93.
24. Seelig A. P-Glycoprotein: One Mechanism, Many Tasks and the Consequences for Pharmacotherapy of Cancers. Front Oncol. 2020; 10(October):1-16.
25. Schumacher M A, Brennan R G. Structural mechanisms of multidrug recognition and regulation by bacterial multidrug transcription factors. Mol Microbiol. 2002; 45(4):885-93.
26. Su X Z, Chen J, Mizushima T, Kuroda T, Tsuchiya T. AbeM, an H+-coupled Acinetobacter baumannii multidrug efflux pump belonging to the MATE family of transporters. Antimicrob Agents Chemother. 2005; 49(10):4362-4.
27. Sun J, Deng Z, Yan A. Bacterial multidrug efflux pumps: Mechanisms, physiology and pharmacological exploitations. Biochem Biophys Res Commun 2014; 453(2):254-67. Available from:
28. Wong K, Ma J, Rothnie A, Biggin P C, Kerr I D. Towards understanding promiscuity in multidrug efflux pumps. Trends Biochem Sci [Internet]. 2014; 39(1):8-16. Available from:
29. Yamaguchi A, Nishino K. Bacterial multidrug exporters methods and protocols. Humana Press. 2018. 1-351 p.
30. Zakrzewska S, Mehdipour A, Malviya V N, Nonaka T, Koepke J, Muenke C, et al. Inward-facing conformation of a multidrug resistance MATE family transporter. Proc Natl Acad Sci US A. 2019; 116(25):12275-84.
31. Zamek-Gliszczynski M J, Hoffmaster K A, Nezasa K I, Tallman M N, Brouwer Yang R, Liu P, Pan D, Zhang P, Bai Z, Xu Y, Wang L, Yan J, Yan Y, Liu X, Yang M. An Investigation on a Novel Anti-tumor Fusion Peptide of FSH33-53-IIKK. J Cancer. 2016 May 24; 7(8):1010-9. doi: 10.7150/jca.14425. PMID: 27313792; PMCID: PMC4910594.
32. Shah, S., Chandra, A., Kaur, A., Sabnis, N., Lacko, A., Gryczynski, Z., et al. (2017). Fluorescence properties of doxorubicin in PBS buffer and PVA films. J Photochem Photobiol B 170, 65-69. doi: 10.1016/j.jphotobiol.2017.03.024.
33. Motlagh, N. S. H., Parvin, P., Ghasemi, F., and Atyabi, F. (2016). Fluorescence properties of several chemotherapy drugs: doxorubicin, paclitaxel and bleomycin. Biomedical Optics Express 7(6), 2400. doi: 10.1364/boe.7.002400.
34. Lu, M., Radchenko, M., Symersky, J., Nie, R., and Guo, Y. (2013). Structural insights into H+-coupled multidrug extrusion by a MATE transporter. Nature Structural & Molecular Biology 20(11), 1310-1317. doi: 10.1038/nsmb.2687.
35. Benatuil, L., Perez, J. M., Belk, J., and Hsieh, C. M. (2010). An improved yeast transformation method for the generation of very large human antibody libraries. Protein Eng Des Sel 23(4), 155-159.

SEQUENCE LEGEND

SEQ ID NO. 1
Amino Acid
T-SNARE EV membrane localized protein SSO2*
Saccharomyces cerevisiae
MSNANPYENNNPYAENYEMQEDLNNAPTGHSDGSDDFVAFMNKINSINANLSRYENIINQIDAQHKDLLT
QVSEEQEMELRRSLDDYISQATDLQYQLKADIKDAQRDGLHDSNKQAQAENCRQKELKLIQDYRIIDSNY
KEESKEQAKRQYTIIQPEATDEEVEAAINDVNGQQIFSQALLNANRRGEAKTALAEVQARHQELLKLEKT
MAELTQLENDMEELVIEQQENVDVIDKNVEDAQQDVEQGVGHTNKAVKSARKARKNKIRCLIICFIIFAI
VVVVVVVPSVVETRK
*Transmembrane spanning amino acid sequences underlined
*bold font aspartate 161 is the last amino acid encoded to generate a
truncated three transmembrane spanning (TMS) protein.
SEQ ID NO. 2
Amino Acid
rhodopsin (BR)*
Haloterrigena turkmenica
MIPELEMYRLGFYVTAVATLVELGWVAKKPAGTRRYYLPAPIVCGTLSLAYFGMSVELLRVMTPSGQPLP
MTRYIDYFVATAIMVAVAGKVAGATRRQLIAIVVLTVGWVGISLGRYFLTGTTVLAATLGTVVVLGALIY
MMVWPVTKRSGETAGERVLLYGKLRNLLILLWVAYLVVGVVSRQGIGLLDAFGGIFAGAYLDIATRIGFG
LLLLRGPDAIAHLIDETGSNGGSGESGDEVTFTESSDDPGTDPDIEPAD
*Transmembrane spanning amino acid sequences underlined
SEQ ID NO. 3
Amino Acid
Glycophorin A transmembrane spanning domain (amino acids 92-114)*
Homo Sapiens
ITLIIFGVMAGVIGTILLISYG
*Transmembrane spanning amino acid sequences underlined
SEQ ID NO. 4
Amino Acid
AbeM protein sequence*
Acinetobacter baumannii
MSNVTSFRSELKQLFHLMLPILITQFAQAGFGLIDTIMAGHLSAADLAAIAVGVGLWIPVMLLESGIMIA
TTPLVAEAKGARNTEQIPVIVRQSLWVAVILGVLAMLILQLMPFFLHVFGVPESLQPKASLFLHAIGLGM
PAVTMYAALRGYSEALGHPRPVTVISLLALVVLIPLNMIFMYGLGPIPALGSAGCGFATSILQWLMLITL
AGYIYKASAYRNTSIFSREDKINLTWVKRILQLGLPIGLAVFFEVSIFSTGALVLSPLGEVFIAAHQVAI
SVTSVLEMIPLSLAIALTIRVGTYYGEKNWASMYQVQKIGLSTAVEFALLTMSFIALGREQIVSVYTQDI
NVVPVAMYLLWFAMAYQLMDALQVSAAGCLRGMQDTQAPMWITLMAYWVIAFPIGLYLARYTDWGVAGVW
LGLIIGLSIACVLLLSRLYLNTKRLSQT
*Transmembrane spanning amino acid sequences underlined
SEQ ID NO. 5
Amino Acid
Chr1 protein ligand anchor
Saccharomyces boulardii
MKVLDLLTVLSASSLLSTFAAAESTATADSTTAASSTASCNPLKTTGCTPDTALATSFSEDESSSSKWFT
DLKHAGEIKYGSDGLSMTLAKRYDNPSLKSNFYIMYGKLEVILKAANGTGIVSSFYLQSDDLDEIDIEWV
GGDNTQFQSNFFSKGDTTTYDRGEFHGVDTPTDKFHNYTLDWAMDKTTWYLDGESVRVLSNTSSEGYPQS
PMYLMMGIWAGGDPDNAAGTIEWAGGETNYNDAPFTMYIEKVIVTDYSTGKKYTYGDQSGSWESIEADGG
SIYGRYDQAQEDFAVLANGGSISSSSTSSSTVSSSASSTVSSSVSSTVSSSASSTVSSSVSSTVSSSSSV
SSSSSTSPSSSTATSSKTLASSSVTTSSSISSFEKQSSSSSKKTVASSSTSESIISSTKTPATVSSTTRS
TVAPTTQQSSVSSDSPVQDKGGVATSSNDVTSSTTQISSKYTSTIQSSSSEASSTNSVQISNGADLAQSL
PREGKLESVLVALLALL
SEQ ID NO. 6
Amino Acid
Sur7 protein ligand anchor
Saccharomyces boulardii
MVKVWNIVLRLVVLLFLAGNTLLLILMIISGATDHYPVNRFYWVQGNTTGIPNAGDETRWTFWGACLQDK
DGSDTCTSNLAPAYPISPVDNENTHINVPHQFISKRDAFYYLTRESFCFFWIALAFVGVSFILYVLTWCS
KMLSEMVLILMSFGFVENTAAVVLQTAASAMAKNAFHDDHRSAQLGASMMGMAWASVELCIVEFILLVEW
SVRARLASTYSIDNSRYRTSSRWNPFHREKEQATDPILTATGPEDMQQSASIVGPSSNANPVTATAATEN
QPKGINFFTIRKSHERPDDVSV
SEQ ID NO. 7
Amino Acid
Nce 102 protein ligand anchor
Saccharomyces cerevisiae
MLALADNILRIINFLFLVISIGLISSLLNTQHRHSSRVNYCMFACAYGIFTDSLYGVFANFIEPLAWPLV
LFTLDFLNFVFTFTAGTVLAVGIRAHSCNNSSYVDSNKITQGSGTRCRQAQAAVAFLYESCAIFLAKTLM
SVENMISNGAFGSGSFSKRRRTGQVGVPTISQV
SEQ ID NO. 8
Amino Acid
C-terminal ligand for binding to NRP1 receptor
Artificial
RPAR
SEQ ID NO. 9
Amino Acid
C-terminal ligand for binding to EGFR receptor
Artificial
AEYLR
SEQ ID NO. 10
Amino Acid
C-terminal FSH ligand for binding to FSHR receptor
Artificial
YTRDLVYKDPARPKIQKTCTFGGG
SEQ ID NO. 11
Amino Acid
Linker protein
Artificial
SGGGGSGG
SEQ ID NO. 12
Amino Acid
ABG xenobiotic-glycoside deconjugating enzyme protein
Agrobacterium sp.
MTDPNTLAARFPGDELFGVATASFQIEGSTKADGRKPSIWDAFCNMPGHVEGRINGDIACDHYNRWEEDL
DLIKEMGVEAYRESLAWPRIIPDGFGPINEKGLDFYDRLVDGCKARGIKTYATLYHWDLPLTLMGDGGWA
SRSTAHAFQRYAKTVMARLGDRLDAVATFNEPWCAVWLSHLYGVHAPGERNMEAALAAMHHINLAHGFGV
EASRHVAPKVPVGLVLNAHSAIPASDGEADLKAAERAFQFHNGAFFDPVEKGEYPAEMMEALGDRMPVVE
AEDLGIISQKLDWWGLNYYTPMRVADDATPGVEFPATMPAPAVSDVKTDIGWEVYAPALHTLVETLYERY
DLPECYITENGACYNMGVENGQVNDQPRLDYYAEHLGIVADLIRDGYPMRGYFAWSLMDNFEWAEGYRMR
FGLVHVDYQTQVRTVKNSGKWYSALASGFPKGNHGVAKG
SEQ ID NO. 13
Amino Acid
Sodium dependent glucose transporter 1 (SGLT1) protein
Homo Sapiens
RTVGGFFLAGRSMVWWPIGASLFASNIGSGHFVGIAGTAAAGGIAIGGYEWNALIFVVVLGWLFVPIYVK
AGVVTMPEYLRKRFGGKRIQVYLSVLSLIVYIFTKISADIFSGAIFIQLAIGLNLYLAIIILLAITALYT
ITGGLAAVIYTDTLQTFIMVVGSFILMGFAFKEVGGYDAFMQKYMEAIPSNISYGNTTIDSSCYRPREDA
FHIFRDPVTGDLPWPGLIFGLSILALWYWCTDQ
SEQ ID NO. 14
Amino Acid
crtYB-Bifunctional lycopene cyclase/phytoene synthase
Rhodotorula diobovata
MGGYDYWLVHARWTIPPSVALWLVFRKLRTWRDVYKTCFLITIAVTATIPWDSYLIRNRIWSYPDSSVVG
PTLFAIPYEEVFFFFVQTYLTSTLYAVLTRPIVHPVLLPRTPSEGRAVRWTGTALLCGVFALAWAKLEEG
GEGTYLALIVGWVAPFLTLLWWVASTHIIAMPRSTLLLAIFAPTFFLWELDARALQRGTWVIEKGTKLGW
DERGLEIEEAVFFLLTNVMIVFGMAACDHCLAVHDLRSYDKGTSSVEPPLHDFGPILINSPDAKQAQRID
DLRAAIEILSVHSKSESTASMVEDGRLRLDLLALYAWCRVCDDLVDNASSVAAAEANIRMISSCLDLLYP
PSTSTPTSHPVRISNEAIAAALPGLSEPERGSFRLLALLPITRPPLDELLDGERTDLSFLAFAGEKAAGG
KPSGGSTSIPSELPIKTDEDLLVYANNVASSVADLCVQLVWAHCATSVPEPEQRAILSAAREMGQALQLV
NIARDVPADLEIHRIYLPGRGLDVPVADLSPDRRELLRRAREMAAHSRGAIERLPREARGGIRAACDVYL
SIGGAVDKALDEGRVLERARVAKGTRAWKAWTAL
SEQ ID NO. 15
Amino Acid
crtI-Phytoene desaturase (neurosporene-forming) protein sequence
Rhodobacter sphaeroides
MPSISPASDADRALVIGSGLGGLAAAMRLGAKGWRVTVIDKLDVPGGRGSSITQEGHREDLGPTIVTVPQ
SLRDLWKTCGRDFDADVELKPIDPFYEVRWPDGSHFTVRQSTEAMKAEVARLSPGDVAGYEKELKDSEKR
YWFGYEDLGRRSMHKLWDLIKVLPTFGMMRADRSVYQHAALRVKDERLRMALSFHPLFIGGDPFNVTSMY
ILVSQLEKEFGVHYAIGGVAAIAAAMAKVIEGQGGSFRMNTEVDEILVEKGTATGVRLASGEVLRAGLVV
SNADAGHTYMRLLRNHPRRRWTDAHVKSRRWSMGLFVWYFGTKGTKGMWPDVGHHTIVNAPRYKGLVEDI
FLKGKLAKDMSLYIHRPSITDPTVAPEGDDTFYALSPVPHLKQAQPVDWQAVAEPYRESVLEVLEQSMPG
IGERIGPSLVFTPETFRDRYLSPWGAGFSIEPRILQSAWFRPHNISEEVANLFLVGAGTHPGAGVPGVIG
SAEVMAKLAPDAPRARREAEPAERLAAE
SEQ ID NO. 16
Amino Acid
crtE Geranylgeranyl pyrophosphate synthase
Agmenellum quadruplicatum
MVVADAHTQGFSLAQYLQEQKTIVETALDQSLVITEPVTIYEAMRYSLLAGGKRLRPILCLAACEMLGGT
AAMAMNTACALEMIHTMSLIHDDLPAMDNDDLRRGKPTNHKVYGEDIAILAGDALLSYAFEYVARTPDVP
AERLLQVIVRLGQAVGAEGLVGGQVVDLESEGKTDVAVETLNFIHTHKTGALLEVCVTAGAILAGAKPEE
VQLLSRYAQNIGLAFQIVDDILDITATAEELGKTAGKDLEAQKVTYPSLWGIEKSQAEAQKLVAEAIASL
EPYGEKANPLKALAEYIVNRKN
SEQ ID NO. 17
Amino Acid
Sur7 protein ligand anchor-truncated
Saccharomyces boulardii
MVKVWNIVLRLVVLLFLAGNTLLLILMIISGATDHYPVNRFYWVQGNTTGIPNAGDETRWTFWG
ACLQDKDGSDTCTSNLAPAYPISPVDNENTHINVPHQFISKRDAFYYLTRESFCFFWIALAFVG
VSFILYVLTWCSKMLSEMVLILMSFGFVENTAAVVLQTAASAMAKNAFHDDHRS
SEQ ID NO. 18
Amino Acid
Sur7/GGGS3-linker/AbeM*
Artificial
MVKVWNIVLRLVVLLFLAGNTLLLILMIISGATDHYPVNRFYWVQGNTTGIPNAGDETRWTFWG
ACLQDKDGSDTCTSNLAPAYPISPVDNENTHINVPHQFISKRDAFYYLTRFSFCFFWIALAFVG
VSFILYVLTWCSKMLSEMVLILMSFGFVENTAAVVLQTAASAMAKNAFHDDHRSAQLGASMMGM
AWASVFLCIVEFILLVEWSVRARLASTYSIDNSRYRTSSRWNPFHREKEQATDPILTATGPEDM
QQSASIVGPSSNANPVTATAATENQPKGINFFTIRKSHERPDDVSVSGGGGSGGGGSGGGGSMS
NVTSFRSELKQLFHLMLPILITQFAQAGFGLIDTIMAGHLSAADLAAIAVGVGLWIPVMLLFSG
IMIATTPLVAEAKGARNTEQIPVIVRQSLWVAVILGVLAMLILQLMPFFLHVFGVPESLQPKAS
LFLHAIGLGMPAVTMYAALRGYSEALGHPRPVTVISLLALVVLIPLNMIFMYGLGPIPALGSAG
CGFATSILQWLMLITLAGYIYKASAYRNTSIFSRFDKINLTWVKRILQLGLPIGLAVFFEVSIF
STGALVLSPLGEVFIAAHQVAISVTSVLFMIPLSLAIALTIRVGTYYGEKNWASMYQVQKIGLS
TAVFFALLTMSFIALGREQIVSVYTQDINVVPVAMYLLWFAMAYQLMDALQVSAAGCLRGMQDT
QAPMWITLMAYWVIAFPIGLYLARYTDWGVAGVWLGLIIGLSIACVLLLSRLYLNTKRLSQT
*Sur7 sequence is normal font, AbeM sequence is in bold font.
Peptide linker is in italics.
SEQ ID NO. 19
Amino Acid
Sur7 truncated/GGGS3-linker/AbeM*
Artificial
MVKVWNIVLRLVVLLFLAGNTLLLILMIISGATDHYPVNRFYWVQGNTTGIPNAGDETRWTFWG
ACLQDKDGSDTCTSNLAPAYPISPVDNFNTHINVPHQFISKRDAFYYLTRESFCFFWIALAFVG
VSFILYVLTWCSKMLSEMVLILMSFGFVENTAAVVLQTAASAMAKNAFHDDHRSSGGGGSGGGG
SGGGGSMSNVTSFRSELKQLFHLMLPILITQFAQAGFGLIDTIMAGHLSAADLAAIAVGVGLWI
PVMLLFSGIMIATTPLVAEAKGARNTEQIPVIVRQSLWVAVILGVLAMLILQLMPFFLHVFGVP
ESLQPKASLFLHAIGLGMPAVTMYAALRGYSEALGHPRPVTVISLLALVVLIPLNMIFMYGLGP
IPALGSAGCGFATSILQWLMLITLAGYIYKASAYRNTSIFSRFDKINLTWVKRILQLGLPIGLA
VFFEVSIFSTGALVLSPLGEVFIAAHQVAISVTSVLFMIPLSLAIALTIRVGTYYGEKNWASMY
QVQKIGLSTAVFFALLTMSFIALGREQIVSVYTQDINVVPVAMYLLWFAMAYQLMDALQVSAAG
CLRGMQDTQAPMWITLMAYWVIAFPIGLYLARYTDWGVAGVWLGLIIGLSIACVLLLSRLYLNT
KRLSQT
*Sur7 sequence is normal font, AbeM sequence is in bold font.
Peptide linker is in italics.
SEQ ID NO. 20
Amino Acid
Linker protein
Artificial
SGGGGSGGGGSGGGGS
SEQ ID NO. 21
Amino Acid
AbeM/p-ESM*
Artificial
MSNVTSFRSELKQLFHLMLPILITQFAQAGFGLIDTIMAGHLSAADLAAIAVGVGLWIPVMLLF
SGIMIATTPLVAEAKGARNTEQIPVIVRQSLWVAVILGVLAMLILQLMPFFLHVFGVPESLQPK
ASLFLHAIGLGMPAVTMYAALRGYSEALGHPRPVTVISLLALVVLIPLNMIFMYGLGPIPALGS
AGCGFATSILQWLMLITLAGYIYKASAYRNTSIFSRFDKINLTWVKRILQLGLPIGLAVFFEVS
IFSTGALVLSPLGEVFIAAHQVAISVTSVLFMIPLSLAIALTIRVGTYYGEKNWASMYQVQKIG
LSTAVFFALLTMSFIALGREQIVSVYTQDINVVPVAMYLLWFAMAYQLMDALQVSAAGCLRGMQ
DTQAPMWITLMAYWVIAFPIGLYLARYTDWGVAGVWLGLIIGLSIACVLLLSRLYLNTKRLSQT
TSQTTTTSIVSSASLQTAITSTLSPATKSSSVVSLQTATTSTLSPTTTSTSSGSTSSDSTA
SEQ ID NO. 22
Amino Acid
p-ESM
Artificial
TSQTTTTSIVSSASLQTAITSTLSPATKSSSVVSLQTATTSTLSPTTTSTSSGSTSSDSTA

1. A method of loading a xenobiotic compound into a Saccharomyces-generated extracellular vesicle (EV), the method comprising:
establishing an EV having an acidic lumenal pH, wherein the EV includes a fusion peptide having a first membrane targeting domain, and said second a proton driven xenobiotic transporter domain; and
generating a pH gradient across the membrane of the EV, wherein a xenobiotic is transported into the lumen of the EV by the transporter.

2. The method of claim 1, wherein said Saccharomyces-generated EV is selected from a Saccharomyces cerevisiae generated EV, or a Saccharomyces boulardii EV.

3. The method of claim 1, wherein said step of establishing comprises incubating the EV in an acidic solution.

4. The method of claim 3, wherein said acidic solution has a pH of approximately 3.

5. The method of claim 1, wherein said lumenal pH is approximately 4.

6. The method of claim 1, wherein said step of generating a pH gradient comprises the step of contacting the EV with a transfer solution having a pH that is higher than the lumenal pH.

7. The method of claim 6, wherein said transfer solution has a pH of approximately 8.

8. The method of claim 1, wherein the xenobiotic is added to the transfer solution prior to generating the pH gradient.

9. The method of claim 1, wherein said proton driven xenobiotic transporter comprises an AbeM protein, or fragment thereof.

10. The method of claim 1, wherein said proton driven xenobiotic transporter is selected from a peptide having an amino acid sequence according to SEQ ID NO. 4, or a sequence having at least 80% sequence homology with SEQ ID NO. 4, or a fragment thereof.

11. The method of claim 1, wherein said membrane targeting domain orients the proton driven xenobiotic transporter to pump a xenobiotics into the EV.

12. The method of claim 1, wherein said membrane targeting domain comprises an EV membrane protein anchor.

13. The method of claim 12, wherein said EV membrane protein anchor comprises a truncated Sur7 protein.

14. The method of claim 12, wherein said EV membrane protein anchor is selected from a peptide having an amino acid sequence according to SEQ ID NO. 17, or a sequence having at least 80% sequence homology with SEQ ID NO. 17, or a fragment thereof.

15. The method of claim 1, wherein said membrane targeting domain comprises a protein extracellular vesicle sorting motif (p-ESM).

16. The method of claim 15, wherein said p-ESM comprises an amino acid sequence according to SEQ ID NO. 22, or fragment thereof.

17. The method of claim 1, wherein said first and said second domains of the fusion peptide are joined by a linker.

18. (canceled)

19. The method of claim 1, wherein said fusion peptide is selected from an amino acid sequence according to SEQ ID NO.'s 19, and 21, or a sequence having at least 80% sequence homology with SEQ ID NO.'s 19, or 21, or a fragment thereof.

20. (canceled)

21. The method of claim 1, wherein the xenobiotic is selected from: a therapeutic compound, a drug, a biologic, a DNA or RNA oligonucleotide, an exonuclease, flavorings, fragrances, food additives, a dye, a probe, a chemical compound, anti-cancer compounds, antibiotics, or conjugated variants, 5-fluorouracil, kanamycin, gentamicin, ofloxacin, ciprofloxacin, erythromycin, chloramphenicol, DAPI, triclosan, acriflavine, Hoechst 33342, daunomycin, doxorubicin, trimethoprim, and/or rhodamine 6G.

22. (canceled)

23. A Saccharomyces-generated extracellular vesicle (EV) produced by the method any of claims 1 to 2.

24-63. (canceled)


SEQUENCE LISTING (22 sequences)

SEQ ID NO: 1 - protein, 295 aa, Saccharomyces cerevisiae
MSNANPYENNNPYAENYEMQEDLNNAPTGHSDGSDDFVAFMNKINSINANLSRYENIINQIDAQHKDLLTQVSEEQEMELRRSLDDYISQATDLQYQLKADIKDAQRDGLHDSNKQAQAENCRQKFLKLIQDYRIIDSNYKEESKEQAKRQYTIIQPEATDEEVEAAINDVNGQQIFSQALLNANRRGEAKTALAEVQARHQELLKLEKTMAELTQLFNDMEELVIEQQENVDVIDKNVEDAQQDVEQGVGHTNKAVKSARKARKNKIRCLIICFIIFAIVVVVVVVPSVVETRK

SEQ ID NO: 2 - protein, 259 aa, Haloterrigena turkmenica
MIPELEMYRLGFYVTAVATLVFLGWVAKKPAGTRRYYLPAPIVCGTLSLAYFGMSVELLRVMTPSGQPLPMTRYIDYFVATAIMVAVAGKVAGATRRQLIAIVVLTVGWVGISLGRYFLTGTTVLAATLGTVVVLGALIYMMVWPVTKRSGETAGERVLLYGKLRNLLILLWVAYLVVGVVSRQGIGLLDAFGGIFAGAYLDIATRIGFGLLLLRGPDAIAHLIDETGSNGGSGESGDEVTFTESSDDPGTDPDIEPAD

SEQ ID NO: 3 - protein, 22 aa, Homo sapiens
ITLIIFGVMAGVIGTILLISYG

SEQ ID NO: 4 - protein, 448 aa, Acinetobacter baumannii
MSNVTSFRSELKQLFHLMLPILITQFAQAGFGLIDTIMAGHLSAADLAAIAVGVGLWIPVMLLFSGIMIATTPLVAEAKGARNTEQIPVIVRQSLWVAVILGVLAMLILQLMPFFLHVFGVPESLQPKASLFLHAIGLGMPAVTMYAALRGYSEALGHPRPVTVISLLALVVLIPLNMIFMYGLGPIPALGSAGCGFATSILQWLMLITLAGYIYKASAYRNTSIFSRFDKINLTWVKRILQLGLPIGLAVFFEVSIFSTGALVLSPLGEVFIAAHQVAISVTSVLFMIPLSLAIALTIRVGTYYGEKNWASMYQVQKIGLSTAVFFALLTMSFIALGREQIVSVYTQDINVVPVAMYLLWFAMAYQLMDALQVSAAGCLRGMQDTQAPMWITLMAYWVIAFPIGLYLARYTDWGVAGVWLGLIIGLSIACVLLLSRLYLNTKRLSQT

SEQ ID NO: 5 - protein, 507 aa, Saccharomyces boulardii
MKVLDLLTVLSASSLLSTFAAAESTATADSTTAASSTASCNPLKTTGCTPDTALATSFSEDFSSSSKWFTDLKHAGEIKYGSDGLSMTLAKRYDNPSLKSNFYIMYGKLEVILKAANGTGIVSSFYLQSDDLDEIDIEWVGGDNTQFQSNFFSKGDTTTYDRGEFHGVDTPTDKFHNYTLDWAMDKTTWYLDGESVRVLSNTSSEGYPQSPMYLMMGIWAGGDPDNAAGTIEWAGGETNYNDAPFTMYIEKVIVTDYSTGKKYTYGDQSGSWESIEADGGSIYGRYDQAQEDFAVLANGGSISSSSTSSSTVSSSASSTVSSSVSSTVSSSASSTVSSSVSSTVSSSSSVSSSSSTSPSSSTATSSKTLASSSVTTSSSISSFEKQSSSSSKKTVASSSTSESIISSTKTPATVSSTTRSTVAPTTQQSSVSSDSPVQDKGGVATSSNDVTSSTTQISSKYTSTIQSSSSEASSTNSVQISNGADLAQSLPREGKLFSVLVALLALL

SEQ ID NO: 6 - protein, 302 aa, Saccharomyces boulardii
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSV

SEQ ID NO: 7 - protein, 173 aa, Saccharomyces cerevisiae
MLALADNILRIINFLFLVISIGLISSLLNTQHRHSSRVNYCMFACAYGIFTDSLYGVFANFIEPLAWPLVLFTLDFLNFVFTFTAGTVLAVGIRAHSCNNSSYVDSNKITQGSGTRCRQAQAAVAFLYFSCAIFLAKTLMSVFNMISNGAFGSGSFSKRRRTGQVGVPTISQV

SEQ ID NO: 8 - protein, 4 aa, synthetic construct
RPAR

SEQ ID NO: 9 - protein, 5 aa, synthetic construct
AEYLR

SEQ ID NO: 10 - protein, 24 aa, synthetic construct
YTRDLVYKDPARPKIQKTCTFGGG

SEQ ID NO: 11 - protein, 8 aa, synthetic construct
SGGGGSGG

SEQ ID NO: 12 - protein, 459 aa, Agrobacterium species
MTDPNTLAARFPGDFLFGVATASFQIEGSTKADGRKPSIWDAFCNMPGHVFGRHNGDIACDHYNRWEEDLDLIKEMGVEAYRFSLAWPRIIPDGFGPINEKGLDFYDRLVDGCKARGIKTYATLYHWDLPLTLMGDGGWASRSTAHAFQRYAKTVMARLGDRLDAVATFNEPWCAVWLSHLYGVHAPGERNMEAALAAMHHINLAHGFGVEASRHVAPKVPVGLVLNAHSAIPASDGEADLKAAERAFQFHNGAFFDPVFKGEYPAEMMEALGDRMPVVEAEDLGIISQKLDWWGLNYYTPMRVADDATPGVEFPATMPAPAVSDVKTDIGWEVYAPALHTLVETLYERYDLPECYITENGACYNMGVENGQVNDQPRLDYYAEHLGIVADLIRDGYPMRGYFAWSLMDNFEWAEGYRMRFGLVHVDYQTQVRTVKNSGKWYSALASGFPKGNHGVAKG

SEQ ID NO: 13 - protein, 243 aa, Homo sapiens
RTVGGFFLAGRSMVWWPIGASLFASNIGSGHFVGIAGTAAAGGIAIGGYEWNALIFVVVLGWLFVPIYVKAGVVTMPEYLRKRFGGKRIQVYLSVLSLIVYIFTKISADIFSGAIFIQLAIGLNLYLAIIILLAITALYTITGGLAAVIYTDTLQTFIMVVGSFILMGFAFKEVGGYDAFMQKYMEAIPSNISYGNTTIDSSCYRPREDAFHIFRDPVTGDLPWPGLIFGLSILALWYWCTDQ

SEQ ID NO: 14 - protein, 594 aa, Rhodotorula diobovata
MGGYDYWLVHARWTIPPSVALWLVFRKLRTWRDVYKTCFLITIAVTATIPWDSYLIRNRIWSYPDSSVVGPTLFAIPYEEVFFFFVQTYLTSTLYAVLTRPIVHPVLLPRTPSEGRAVRWTGTALLCGVFALAWAKLEEGGEGTYLALIVGWVAPFLTLLWWVASTHIIAMPRSTLLLAIFAPTFFLWELDARALQRGTWVIEKGTKLGWDFRGLEIEEAVFFLLTNVMIVFGMAACDHCLAVHDLRSYDKGTSSVFPPLHDFGPILINSPDAKQAQRIDDLRAAIEILSVHSKSFSTASMVFDGRLRLDLLALYAWCRVCDDLVDNASSVAAAEANIRMISSCLDLLYPPSTSTPTSHPVRISNEAIAAALPGLSEPERGSFRLLALLPITRPPLDELLDGFRTDLSFLAFAGEKAAGGKPSGGSTSIPSELPIKTDEDLLVYANNVASSVADLCVQLVWAHCATSVPEPEQRAILSAAREMGQALQLVNIARDVPADLEIHRIYLPGRGLDVPVADLSPDRRELLRRAREMAAHSRGAIERLPREARGGIRAACDVYLSIGGAVDKALDEGRVLERARVAKGTRAWKAWTAL

SEQ ID NO: 15 - protein, 518 aa, Rhodobacter sphaeroides
MPSISPASDADRALVIGSGLGGLAAAMRLGAKGWRVTVIDKLDVPGGRGSSITQEGHRFDLGPTIVTVPQSLRDLWKTCGRDFDADVELKPIDPFYEVRWPDGSHFTVRQSTEAMKAEVARLSPGDVAGYEKFLKDSEKRYWFGYEDLGRRSMHKLWDLIKVLPTFGMMRADRSVYQHAALRVKDERLRMALSFHPLFIGGDPFNVTSMYILVSQLEKEFGVHYAIGGVAAIAAAMAKVIEGQGGSFRMNTEVDEILVEKGTATGVRLASGEVLRAGLVVSNADAGHTYMRLLRNHPRRRWTDAHVKSRRWSMGLFVWYFGTKGTKGMWPDVGHHTIVNAPRYKGLVEDIFLKGKLAKDMSLYIHRPSITDPTVAPEGDDTFYALSPVPHLKQAQPVDWQAVAEPYRESVLEVLEQSMPGIGERIGPSLVFTPETFRDRYLSPWGAGFSIEPRILQSAWFRPHNISEEVANLFLVGAGTHPGAGVPGVIGSAEVMAKLAPDAPRARREAEPAERLAAE

SEQ ID NO: 16 - protein, 302 aa, Agmenellum quadruplicatum
MVVADAHTQGFSLAQYLQEQKTIVETALDQSLVITEPVTIYEAMRYSLLAGGKRLRPILCLAACEMLGGTAAMAMNTACALEMIHTMSLIHDDLPAMDNDDLRRGKPTNHKVYGEDIAILAGDALLSYAFEYVARTPDVPAERLLQVIVRLGQAVGAEGLVGGQVVDLESEGKTDVAVETLNFIHTHKTGALLEVCVTAGAILAGAKPEEVQLLSRYAQNIGLAFQIVDDILDITATAEELGKTAGKDLEAQKVTYPSLWGIEKSQAEAQKLVAEAIASLEPYGEKANPLKALAEYIVNRKN

SEQ ID NO: 17 - protein, 182 aa, Saccharomyces boulardii
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRS

SEQ ID NO: 18 - protein, 766 aa, synthetic construct
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSAQLGASMMGMAWASVFLCIVEFILLVFWSVRARLASTYSIDNSRYRTSSRWNPFHREKEQATDPILTATGPEDMQQSASIVGPSSNANPVTATAATENQPKGINFFTIRKSHERPDDVSVSGGGGSGGGGSGGGGSMSNVTSFRSELKQLFHLMLPILITQFAQAGFGLIDTIMAGHLSAADLAAIAVGVGLWIPVMLLFSGIMIATTPLVAEAKGARNTEQIPVIVRQSLWVAVILGVLAMLILQLMPFFLHVFGVPESLQPKASLFLHAIGLGMPAVTMYAALRGYSEALGHPRPVTVISLLALVVLIPLNMIFMYGLGPIPALGSAGCGFATSILQWLMLITLAGYIYKASAYRNTSIFSRFDKINLTWVKRILQLGLPIGLAVFFEVSIFSTGALVLSPLGEVFIAAHQVAISVTSVLFMIPLSLAIALTIRVGTYYGEKNWASMYQVQKIGLSTAVFFALLTMSFIALGREQIVSVYTQDINVVPVAMYLLWFAMAYQLMDALQVSAAGCLRGMQDTQAPMWITLMAYWVIAFPIGLYLARYTDWGVAGVWLGLIIGLSIACVLLLSRLYLNTKRLSQT

SEQ ID NO: 19 - protein, 646 aa, synthetic construct
MVKVWNIVLRLVVLLFLAGNTLLLILMIISGATDHYPVNRFYWVQGNTTGIPNAGDETRWTFWGACLQDKDGSDTCTSNLAPAYPISPVDNFNTHINVPHQFISKRDAFYYLTRFSFCFFWIALAFVGVSFILYVLTWCSKMLSEMVLILMSFGFVFNTAAVVLQTAASAMAKNAFHDDHRSSGGGGSGGGGSGGGGSMSNVTSFRSELKQLFHLMLPILITQFAQAGFGLIDTIMAGHLSAADLAAIAVGVGLWIPVMLLFSGIMIATTPLVAEAKGARNTEQIPVIVRQSLWVAVILGVLAMLILQLMPFFLHVFGVPESLQPKASLFLHAIGLGMPAVTMYAALRGYSEALGHPRPVTVISLLALVVLIPLNMIFMYGLGPIPALGSAGCGFATSILQWLMLITLAGYIYKASAYRNTSIFSRFDKINLTWVKRILQLGLPIGLAVFFEVSIFSTGALVLSPLGEVFIAAHQVAISVTSVLFMIPLSLAIALTIRVGTYYGEKNWASMYQVQKIGLSTAVFFALLTMSFIALGREQIVSVYTQDINVVPVAMYLLWFAMAYQLMDALQVSAAGCLRGMQDTQAPMWITLMAYWVIAFPIGLYLARYTDWGVAGVWLGLIIGLSIACVLLLSRLYLNTKRLSQT

SEQ ID NO: 20 - protein, 16 aa, synthetic construct
SGGGGSGGGGSGGGGS

SEQ ID NO: 21 - protein, 509 aa, synthetic construct
MSNVTSFRSELKQLFHLMLPILITQFAQAGFGLIDTIMAGHLSAADLAAIAVGVGLWIPVMLLFSGIMIATTPLVAEAKGARNTEQIPVIVRQSLWVAVILGVLAMLILQLMPFFLHVFGVPESLQPKASLFLHAIGLGMPAVTMYAALRGYSEALGHPRPVTVISLLALVVLIPLNMIFMYGLGPIPALGSAGCGFATSILQWLMLITLAGYIYKASAYRNTSIFSRFDKINLTWVKRILQLGLPIGLAVFFEVSIFSTGALVLSPLGEVFIAAHQVAISVTSVLFMIPLSLAIALTIRVGTYYGEKNWASMYQVQKIGLSTAVFFALLTMSFIALGREQIVSVYTQDINVVPVAMYLLWFAMAYQLMDALQVSAAGCLRGMQDTQAPMWITLMAYWVIAFPIGLYLARYTDWGVAGVWLGLIIGLSIACVLLLSRLYLNTKRLSQTTSQTTTTSIVSSASLQTAITSTLSPATKSSSVVSLQTATTSTLSPTTTSTSSGSTSSDSTA

SEQ ID NO: 22 - protein, 61 aa, synthetic construct
TSQTTTTSIVSSASLQTAITSTLSPATKSSSVVSLQTATTSTLSPTTTSTSSGSTSSDSTA